(19) United States
(12) Patent Application Publication
(10) Pub. No.: US 2004/0247525 A1
(43) Pub. Date: Dec. 9, 2004

(54) Brain progenitor cell-based assay, and related methods and compositions

(76) Inventors: Rene Hen, Tenafly, NJ (US); Luca Santarelli, New York, NY (US); Michael Saxe, New York, NY (US)

Correspondence Address: Cooper and Dunham LLP, 1185 Avenue of the Americas, New York, NY, 10036, US

(21) Appl. No.: 10/764,068

(22) Filed: Jan. 22, 2004

Related U.S. Application Data
(60) Provisional application No. 60/526,190, filed on Dec. 1, 2003.
(60) Provisional application No. 60/442,081, filed on Jan. 23, 2003.

(51) Int. Cl. A61K 49/00

(52) U.S. Cl. 424/9.2; 800/9

(57) ABSTRACT

This invention provides methods for determining whether an agent increases brain progenitor cell division in a subject. Furthermore, this invention provides methods for treating anxiety, depression, a cognitive disorder or a neuro-degenerative disorder or inhibiting the onset of anxiety, depression or a cognitive disorder by administering to an afflicted subject a therapeutically or prophylactically effective amount of the agent. Finally, this invention provides methods for treating anxiety, depression, a cognitive disorder or a neuro-degenerative disorder or inhibiting the onset of anxiety, depression or a cognitive disorder by administering to an afflicted subject a therapeutically or prophylactically effective amount of Hh-Ag 1.1, Hh-Ag 1.2, Hh-Ag 1.3, or a derivative of Hh-Ag 1.1, Hh-Ag 1.2 or Hh-Ag 1.3.

This application claims the benefit of copending U.S. Provisional Applications No. 60/526,190, filed Dec. 1, 2003, and No. 60/442,081 filed Jan. 23, 2003, the contents of which are hereby incorporated by reference. This invention was made with support under United States Government NIDA Grant R01 DA09862 and NIMH Grants PO1 MH48125 and P50 MH50733.

This invention was made with support under United States Government NIDA Grant R01 DA09862 and NIMH Grants PO1 MH48125 and P50 MH50733.

Throughout this application, various publications are referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

Anxiety Disorders and Neurogenesis

Both depression and anxiety disorders are common public health problems, with 10-20% lifetime prevalence (Wong and Licinio, 2001), yet the mechanisms underlying their pathophysiology are still poorly understood. Following the serendipitous discovery of antidepressant drugs (“Ads”) in the early 1950's, the study of their mechanism of action has been used to gain insight into the pathophysiology of anxiety and depression.

To date, most ADs have been shown to increase levels of the monoamines serotonin (5-HT) and/or noradrenaline (NA). This observation suggests that biochemical imbalances within these neurotransmitter systems may underlie the pathogenesis of these disorders, a theory also known as “the monoaminergic hypothesis of depression”. However, while ADs produce a rapid increase in extracellular levels of 5-HT and NA, the onset of an appreciable clinical effect usually takes at least three to four weeks (Wong and Licinio, 2001). This delay suggests that slow neurochemical and structural changes take place within the limbic target areas of monoaminergic projections, and these changes may counteract neuropathological alterations that initiate or perpetuate anxiety and depressive disorders. Indeed, recent post mortem and brain imaging studies have demonstrated consistent findings of atrophy or loss of neurons in the prefrontal cortex and hippocampus of both depressed and anxious patients (Bremner et al., 1997; Duman and Charney, 1999; Gurvits et al., 1996; Shah et al., 1998; Sheline et al., 1996; Stein et al., 1997), and some of these alterations may be reversed by ADs (Czeh et al., 2001; Moore et al., 2000). In addition, stress, an environmental factor capable of precipitating depressive episodes in humans and used to model depression in laboratory animals, causes cell death, dendritic shrinkage, and decreased levels of neurotrophins (e.g., BDNF) within the hippocampus (Duman et al., 1997; McEwen, 1999; Sapolsky, 1992), as well as a reduction in hippocampal granule cell neurogenesis (Gould et al., 1998). Although it is unclear whether any of these events contributes to the pathogenesis of depression, the recent observation that adult hippocampal neurogenesis is decreased by stress and increased by chronic antidepressants (Chen et al., 2000; Gould and Tanapat, 1999; Gould et al., 1998; Malberg et al., 2000; Manev et al., 2001) suggests that this process may be involved in both the pathogenesis and treatment of mood disorders.

Adult neurogenesis consists of the production of new neurons within the brain of an adult organism. In the 1960's, using tritiated thymidine autoradiography, Altman and Das described the generation of new cells in a variety of brain structures in the adult rat and cat, including the olfactory bulb, hippocampus, and cerebral cortex (Altman, 1962; Altman and Das, 1965; Altman and Das, 1966). Later studies employed ultrastructural analysis of tritiated thymidine-labeled cells (Kaplan and Hinds, 1977), as well as immunohistochemical recognition of cell type-specific markers in combination with the DNA synthesis marker bromodeoxyuridine (BrdU) (Kuhn et al., 1996; Seki and Arai, 1995), to identify the phenotype of newly-generated cells. These findings provided convincing evidence that adult-generated brain cells can differentiate into neurons, and showed this phenomenon to be primarily confined to two discrete areas of the brain: the subventricular zone, and the subgranular zone of the dentate gyrus (Garcia-Verdugo et al., 1998).

In the hippocampus of both rodents and primates, adult-generated neuronal cells appear to arise from progenitor cells in the subgranular zone and migrate into the granule cell layer, where they differentiate into granular neurons (Gould and Gross, 2002). Recently, these cells were shown to be capable of functional integration into the hippocampal circuitry, as evidenced by their responsiveness to stimulation of the perforant path and their ability to extend axonal projections to appropriate target areas (van Praag et al., 2002). Although the function of newly-generated cells in the adult hippocampus is still unclear, it has been suggested that young granule cells constitute a distinct population exhibiting a greater degree of plasticity than mature neurons (Gould and Gross, 2002). Accordingly, various factors that have been shown to exert a modulatory influence on adult hippocampal neurogenesis also alter hippocampal-dependent functions and plasticity. For instance, manipulations that increase neurogenesis, such as an enriched environment and physical activity, are associated with improved memory and enhanced long-term synaptic plasticity in the hippocampus (Duffy et al., 2001; Kempermann et al., 1997; Nilsson et al., 1999; Pham et al., 1999; Williams et al., 2001). Conversely, stress or other manipulations that produce a decrease in hippocampal neurogenesis, are associated with memory impairment and disruption of hippocampal plasticity (McEwen, 1999).

Notwithstanding the above assertions, there is no known requirement that brain progenitor cell division take place in order for an anti-depressant to achieve its desired result.

Hedgehog Pathway

The hedgehog (Hh) signaling pathway is vital to animal development as it mediates the differentiation of multiple cell types during embryogenesis. In adults, Hh signaling can be activated to facilitate tissue maintenance and repair. Moreover, stimulation of the Hh pathway has shown therapeutic efficacy in models of Parkinson's disease and diabetic neuropathy. Small-molecule modulators of Hedgehog signaling have been characterized (Frank-Kamenetsky et al., 2002).

### SUMMARY OF THE INVENTION

This invention provides a method for determining whether an agent increases brain progenitor cell division comprising: (i) administering the agent to a non-human subject; and (ii) determining whether the resulting brain progenitor cell division in the subject is greater than that in a subject to which the agent was not administered, thereby determining whether the agent increases brain progenitor cell division.

This invention further provides a method for treating anxiety, depression, a cognitive disorder or a neuro-degenerative disorder by administering to an afflicted subject a therapeutically effective amount of an agent determined to have the ability to increase brain progenitor cell division, wherein such ability is determined by a method comprising (i) administering the agent to a non-human subject, and (ii) determining whether the resulting brain progenitor cell division in the subject is greater than that in a subject to which the agent was not administered.

This invention further provides a method for inhibiting the onset of anxiety, depression or a cognitive disorder by administering to a subject in need thereof a prophylactically effective amount of an agent determined as having the ability to increase brain progenitor cell division, wherein such ability is determined by a method comprising (i) administering the agent to a non-human subject, and (ii) determining whether the resulting brain progenitor cell division in the subject is greater than that in a subject to which the agent was not administered.

This invention further provides a composition comprising (a) a pharmaceutically acceptable carrier, and (b) an agent determined as having the ability to increase brain progenitor cell division, wherein such ability is determined by a method comprising (i) administering the agent to a non-human subject, and (ii) determining whether the resulting brain progenitor cell division in the subject is greater than that in a subject to which the agent was not administered.

This invention further provides an article of manufacture comprising a packaging material having therein an agent determined as having the ability to increase brain progenitor cell division, and a label indicating a use of the agent for inhibiting the onset of anxiety, depression or a cognitive disorder in a subject, wherein such ability is determined by a method comprising (i) administering the agent to a non-human subject, and (ii) determining whether the resulting brain progenitor cell division in the subject is greater than that in a subject to which the agent was not administered.

This invention further provides an article of manufacture comprising a packaging material having therein an agent determined as having the ability to increase brain progenitor cell division, and a label indicating a use of the agent for treating anxiety, depression, a cognitive disorder or a neurodegenerative disorder in a subject, wherein such ability is determined by a method comprising (i) administering the agent to a non-human subject, and (ii) determining whether the resulting brain progenitor cell division in the subject is greater than that in a subject to which the agent was not administered.

This invention further provides a method for treating anxiety, depression, a cognitive disorder or a neuro-degenerative disorder by administering to an afflicted subject a therapeutically effective amount of Hh-Ag 1.1, Hh-Ag 1.2, Hh-Ag 1.3, or a derivative of Hh-Ag 1.1, Hh-Ag 1.2 or Hh-Ag 1.3.

This invention further provides a method for inhibiting the onset of anxiety, depression or a cognitive disorder by administering to a subject in need thereof a prophylactically effective amount of Hh-Ag 1.1, Hh-Ag 1.2, Hh-Ag 1.3, or a derivative of Hh-Ag 1.1, Hh-Ag 1.2 or Hh-Ag 1.3.

This invention further provides a composition comprising (a) a pharmaceutically acceptable carrier, and (b) Hh-Ag 1.1, Hh-Ag 1.2, Hh-Ag 1.3, or a derivative of Hh-Ag 1.1, Hh-Ag 1.2 or Hh-Ag 1.3.

This invention further provides an article of manufacture comprising a packaging material having therein Hh-Ag 1.1, Hh-Ag 1.2, Hh-Ag 1.3, or a derivative of Hh-Ag 1.1, Hh-Ag 1.2 or Hh-Ag 1.3 and a label indicating a use of Hh-Ag 1.1, Hh-Ag 1.2, Hh-Ag 1.3, or a derivative of Hh-Ag 1.1, Hh-Ag 1.2 or Hh-Ag 1.3 for inhibiting the onset of anxiety, depression or a cognitive disorder in a subject.

Finally, this invention provides an article of manufacture comprising a packaging material having therein Hh-Ag 1.1, Hh-Ag 1.2, Hh-Ag 1.3, or a derivative of Hh-Ag 1.1, Hh-Ag 1.2 or Hh-Ag 1.3 and a label indicating a use of Hh-Ag 1.1, Hh-Ag 1.2, Hh-Ag 1.3, or a derivative of Hh-Ag 1.1, Hh-Ag 1.2 or Hh-Ag 1.3 for treating anxiety, depression, a cognitive disorder or a neurodegenerative disorder in a subject.

### BRIEF DESCRIPTION OF THE FIGURES

FIGS. 1A-1C Behavioral Effects of Antidepressant Treatment: The Novelty-Suppressed Feeding Paradigm (NSF).

(1A) The NSF maze consists of a rectangular, brightly lit open field filled with sawdust. The center is rendered more aversive by placement of a circular white platform where a pellet of food is positioned.

(1B) A 28-day treatment with antidepressants resulted in a decreased latency to feed, while a 5-day treatment was ineffective. Haloperidol, a neuroleptic compound devoid of antidepressant activity in humans, was ineffective in this test (mean±SEM percentage of vehicle control latency). Unpaired t tests between vehicle (V) and fluoxetine (F), imipramine (I), desipramine (D) or haloperidol (H); *=P<0.05, **=P<0.01 (n=13-15 mice per group).

(1C) Drug effects on 5 min. home cage (h.c.) food consumption (mean±SEM). None of the drugs tested produced a significant change in h.c. food consumption, indicating that the changes seen in (B) are not attributable to enhanced feeding drive.

FIGS. 2A-2D Chronic Fluoxetine Treatment Increases BrdU Uptake and Neurogenesis in the Dentate Gyrus.

(2A) Mice were administered fluoxetine (F) or vehicle (V) for 5, 11, or 28 days and then injected with BrdU for analysis of cell proliferation. No significant effect of fluoxetine was observed after 5 days; however, the number of BrdU-positive cells was significantly increased after 11 and 28 days of treatment (mean±SEM percentage of BrdU-positive cells in vehicle mice; Fisher post-hoc analysis). ANOVA revealed effects of duration of fluoxetine treatment, F_{2,30}=7.2, P=0.026, drug treatment, F_{1,30}=11.5, P=0.0019, and interaction between the two, F_{2,30}=7.2, P=0.026 (n=7-10 mice per group).

(2B) BrdU-immunoreactivity in the dentate gyrus. Mice were treated with either vehicle or fluoxetine for 28 days and sacrificed 24 hours after injection with BrdU. Cell counts were made in the granule cell layer (GCL) and in the subgranular zone (SGZ)—a 50 μm thick lamina at the boundary of the GCL and the hilus. Scale bar, 200 μm.

(2C-2D) Confocal micrographs (40×magnification, zoom of 4) show cells that have acquired a neuronal or astroglial phenotype 28 days after incorporation of BrdU. Immunofluorescent double labeling of BrdU (Green) and NeuN or GFAP (red, C and D). Scale bar, 10 μm.

FIGS. 3A-3C Requirement of 5-HT_{1A} Receptors for the Effects of Fluoxetine on Anxiety-Related Behaviors and Dentate Gyrus Neurogenesis.

(3A) Behavioral effects of chronic antidepressant treatment in wild type (+/+) and 5-HT_{1A} knockout (−/−) mice in the NSF paradigm. Fluoxetine (F), imipramine (I), and desipramine (D) all significantly decreased the latency to feed compared to vehicle controls (V) in +/+, whereas only imipramine and desipramine were effective in −/− mice (mean±SEM percentage of vehicle control latency). ANOVA found an effect of AD treatment, F_{3,77}=5.8, P=0.0012, of genotype, F_{1,77}=33.3, P<0.0001, and an interaction between the two, F_{3,77}=6.2, P=0.0008 (n=10-15 mice per group). Fisher post-hoc tests revealed significant differences between vehicle (V) and treatment, as indicated by *, P<0.05 or **, P<0.01.

(3B-C) Effect of chronic antidepressant treatment on BrdU uptake and survival of BrdU positive cells. At the end of 4 weeks of treatment with F, I, or V, animals were injected with BrdU and sacrificed either 24 hours or 28 days later. BrdU-positive -cells were counted in the SGZ and GCL of the dentate gyrus. While I caused a significant (˜2-fold) increase in the number of BrdU positive cells in both genotypes, F was effective only in +/+ mice. (B). ANOVA revealed a significant effect of AD treatment, F_{2,30}=9.4, P=0.0006, and an interaction between drug and genotype, ANOVA, F_{2,30}=3.2, P=0.05 (n=6-7 mice per group). Significant differences between vehicle (V) and fluoxetine (F) or imipramine (I) were calculated by Fisher post-hoc test. (C) A significant proportion of cells generated at the end of the antidepressant treatment survived ≧28 days; differences between treatment groups were maintained. Effect of ADs treatment, ANOVA, F_{2,16}=10.6, P=0.001, genotype, ANOVA, F_{1,16}=11.9, P=0.032, and interaction between the effect of treatment and genotype, ANOVA, F_{2,16}=6.0, P=0.01 (n=4-5 mice per group). Significant differences (Fisher post-hoc) between vehicle (V) and fluoxetine (F) or imipramine (I) are indicated by *, P<0.05 or **, P<0.01.

FIGS. 4A-4B Direct Stimulation of 5-HT_{1A} Receptors Causes Antidepressant-like Responses and Enhances Cell Proliferation in the SGZ.

(4A) In the NSF test, 28-day administration of the 5-HT_{1A} receptor agonist 8-OH-DPAT (DPAT) delivered by osmotic mini pumps (1 mg/kg/day) resulted in a significant decrease in the latency to feed of wild types (+/+), but not 5-HT_{1A} knockouts (−/−) (mean±SEM percentage of sham +/+ latency). Significant differences between sham and DPAT-treated animals were calculated by a planned Fisher post hoc test (n=11-20 mice per group,).

(4B) Following injection of BrdU at the end of the treatment, +/+ mice, but not −/− mice showed DPAT-induced increases in BrdU-labeled cells in the SGZ (mean±SEM number of BrdU-positive cells). ANOVA revealed an effect of DPAT treatment, F_{1,28}=4.3, P=0.046, and an interaction between treatment and genotype, F_{1,28}=5.8, P=0.02. Differences between sham and DPAT-treated mice were assessed by Fisher post-hoc analysis (n=7-9 mice per group).

FIGS. 5A-5D X-ray Treatment: Ablation of Cell Proliferation in the Dentate Gyrus.

(5A) We devised a means to specifically irradiate the proliferative area of the hippocampus (SGZ) while a lead shield protected the rest of the body of the animal, including other proliferative structures such as the subventricular zone (SVZ). The schematic diagram shows the position of the opening in the lead shield, measuring 3.2 mm rostral from the interaural line, which exposes a portion of the brain encompassing the hippocampal formation to the X-ray source.

(5B) 8 weeks after X-irradiation, a stripe-shaped area of alopecia became visible on the head of the animals, showing the restricted localization where x-rays had been delivered. Compare irradiated (left) with sham (right).

(5C) Experimental design to determine the effect of irradiation on cell proliferation and antidepressant-like behavior in mice treated with chronic fluoxetine or vehicle. A 5 Gy dose of X-ray was delivered on days 1, 4, and 8, and mice were concurrently treated with fluoxetine or vehicle before behavioral testing in the NSF on day 28. Cell proliferation was assessed after injection of BrdU at one of three time points (11, 16 or 27 days after the beginning of the ADs treatment).

(5D) The irradiation resulted in drastically reduced levels of cell proliferation in the subgranular zone (SGZ) of mice treated with either fluoxetine or vehicle at all three time different latencies from the irradiation (mean±SEM percentage of S, V BrdU-positive cells). Irradiation had no effect on cell proliferation in the subventricular zone (SVZ), indicating that use of the lead shield allowed specific targeting of X-rays to the hippocampus.

FIGS. 6A-6D X-ray Suppresses Behavioral Response to Antidepressants in NSF and CUS paradigms.

(6A) Sham and irradiated mice were treated with fluoxetine (F), imipramine (I), or vehicle (V) for 28 days before testing in the NSF test. Although irradiation resulted in no change in the latency to feed of vehicle-treated mice, this procedure blocked the anxiolytic-like effects of antidepressants (mean±SEM percentage of latency of V, sham mice). ANOVA revealed a significant effect of ADs treatment, F_{2,176}=9.3, P=0.0001, X-irradiation, F_{1,176}=9.1, P=0.0025, and an interaction between the two, F_{2,176}=3.9, P=0.02 (n=25-35 mice per group). Fisher post-hoc analysis found significant differences between vehicle (V) and fluoxetine (F) or imipramine (I) in the sham treated group.

(6B) No significant changes in feeding drive, as measured by the amount of food consumed over 5 minutes in the home cage (h.c.) (mean±SEM of food eaten during 5 min.) were caused by either X-ray or drug treatment.

(6C-D) Sham and irradiated mice were subjected to CUS for five weeks, and treated with fluoxetine or vehicle during the last three weeks. Coat state and latency of grooming were measured during the sixth week. (6C) Irradiation prevented the improvement of the state of the coat in the fluoxetine-treated mice, while it had no effect in the vehicle-treated mice (mean±SEM of coat score). ANOVA revealed a significant interaction between X-irradiation and fluoxetine treatment, F_{1,24}=4.6, P=0.044 (n=6-8 mice per group). Fisher post-hoc test revealed significant differences between the fluoxetine-treated sham group and all the others. (6D) In the grooming test, fluoxetine caused a decrease in the latency to groom in sham animals and X-irradiation blocked this effect (mean±SEM of grooming latency). ANOVA revealed a significant main effect of fluoxetine, F_{1,24}=6.4, P=0.02, and an interaction between fluoxetine treatment and X-irradiation, F_{1,24}=4.5, P=0.043 (n=6-8 mice per group). Fisher post-hoc analyses showed significant differences between the fluoxetine-treated sham group and all the others.

FIGS. 7A-7D X-ray Does Not Affect Neuroendocrine Response to Stress, Cued Fear Conditioning, or CA3/CA1 Physiology.

(7A) Serum corticosterone levels 24 hours before and 15 minutes after open field stress are comparable in sham and irradiated mice (mean±SEM of serum concentration; n=15 per group).

(7B) Cued fear conditioning leads to a similar fear response in both sham and irradiated mice. The amount of freezing was measured for 20 sec before and for 20 sec during the presentation of the tone (mean±SEM of the percentage of time spent freezing; n=15 mice per group).

(7C) Sham (open circles) and X-irradiated (closed circles) mice showed similar field input-output relations in Shaffer collateral/CA1 connections (mean±SEM of fEPSP slope; n=8).

(7D) Summary graphs of LTP shows no difference between Sham and X-irradiated mice (mean±SEM of percentage of baseline fEPSP slope; n=8). Arrows indicate the delivery of the theta-burst stimulation.

### DETAILED DESCRIPTION OF THE INVENTION

Definitions

As used in this application, except as otherwise expressly provided herein, each of the following terms shall have the meaning set forth below.

As used herein, “administering” shall mean delivering in a manner which is effected or performed using any of the various methods and delivery systems known to those skilled in the art. Administering can be performed, for example, intravenously, orally, via implant, transmucosally, transdermally, intramuscularly, or subcutaneously. “Administering” can also be performed, for example, once, a plurality of times, and/or over one or more extended periods.

As used herein, “agent” shall include, without limitation, an organic compound, a nucleic acid, a polypeptide, a lipid, and a carbohydrate. Agents include, for example, agents which are known with respect to structure and/or function, and those which are not known with respect to structure or function. In a particular embodiment, an agent is known to have a given structure and effect in connection with a non-neurological disorder, such as depression, but is not known to have a given effect in connection with a neurological disorder.

As used herein, “antibody” shall include, by way of example, both naturally occurring and non-naturally occurring antibodies. Specifically, this term includes polyclonal and monoclonal antibodies, and antigen-binding fragments thereof. Furthermore, this term includes chimeric antibodies and wholly synthetic antibodies, and antigen-binding fragments thereof.

As used herein, “inhibiting” the onset of a disorder shall mean either lessening the likelihood of the disorder's onset, or preventing the onset of the disorder entirely. In the preferred embodiment, inhibiting the onset of a disorder means preventing its onset entirely.

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “pharmaceutically acceptable carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

As used herein, the term “protein” (as with polypeptide) means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

As used herein, “treating” a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

As used herein, “subject” shall mean any animal, such as a non-human primate, mouse, rat, guinea pig, dog, cat, or rabbit.

As used herein, “Hh-Ag 1.1” shall refer to a compound with the following structure:

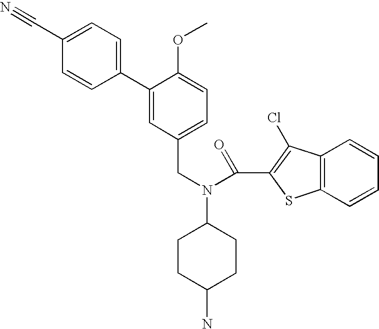

As used herein, “Hh-Ag 1.2” shall refer to a compound with the following structure:

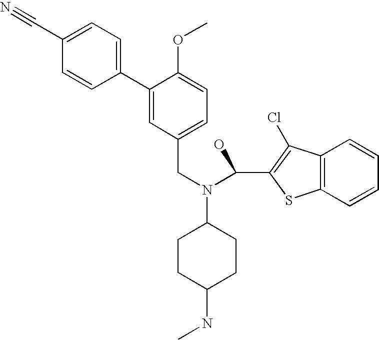

As used herein, “Hh-Ag 1.3” shall refer to a compound with the following structure:

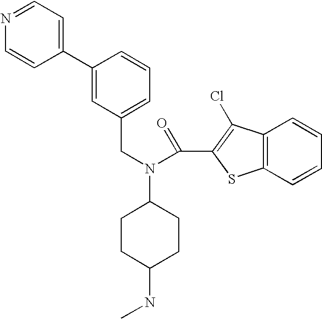

As used herein, “a derivative of Hh-Ag 1.1, Hh-Ag 1.2 or Hh-Ag 1.3” shell refer to a compound with the following structure:

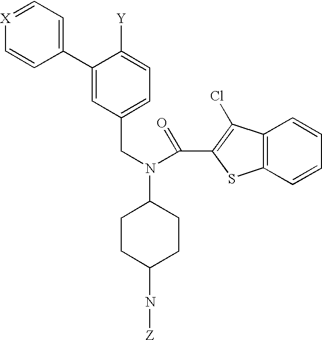

X═C or N≡C—(CH₂)₈—C, where 0≦a≦5

Y═H or —O—(CH2)_{b}—CH₃, where 0≦b≦5

Z═H, CH₃— or CH₃ (CH₂)_{c}—, where 1≦c≦5, and where the

derivative is not Hh-Ag 1.1, Hh-Ag 1.2-or Rh-Ag 1.3.

Embodiments of the Invention

This invention provides a method for determining whether an agent, known to upregulate the sonic hedgehog pathway, increases brain progenitor cell division comprising: (i) administering the agent to e non-human subject; and (ii) determining whether the resulting brain progenitor cell division in the subject is greater than that in a subject to which the agent was not administered, thereby determining whether the agent increases brain progenitor cell division.

In one embodiment of the instant method, the method comprises the steps of (a) administering the agent to the subject for a suitable duration of time (which can include repeated administration if necessary), (b) administering to the subject a compound which is a marker of cell division, (c) sacrificing the subject after a suitable period of time, (d) quantitatively determining incorporation of the compound in the subject's brain tissue; and (e) comparing the amount so determined with the amount of compound in the brain tissue of a subject to which the agent was not administered,

The agent's ability to increase brain progenitor cell division is indicated when the amount of compound in the brain tissue of the subject to which the agent was administered is greater (e.g. by 1%, 5%, 10%, 25%, 50%, 100%, 200% or greater), than the amount of compound in the brain tissue of the subject to which the agent was not administered.

Step (d) can comprise, for example, the steps of formalin profusion (approximately four days), sectioning the brain tissue, staining the tissue sections with anti-BRDU antibody, and counting the cells labeled with antibody.

In another embodiment of this method, the method comprises the steps of (a) administering the agent to the subject (e.g. a mouse, rat, or non-human primate) for a suitable duration (e.g., once, a plurality of times, or continuously over, for example, a period of several days, one week, two weeks, or one month), (b) sacrificing the subject after a suitable period of time, (c) determining, ex vivo, the amount of protein and/or nucleic acid in the subject's brain tissue indicative of brain progenitor cell division, and (d) comparing the amount so determined with the amount of compound in the brain tissue of a subject to which the agent was not administered, as determined ex vivo. The agent's ability to increase brain progenitor cell division is indicated when the amount of compound in the brain tissue of the subject to which the agent was administered is greater than the amount of compound in the brain tissue of the subject to which the agent was not administered.

Step (c) can comprise, for example, extracting mRNA indicative of progenitor cell division from the brain tissue (e.g. hippocampus), and using PCR (real-time PCR) to quantitate the mRNA. Such mRNA includes, without limitation, mRNA encoding Ki-67, a cyclin, a nestin, a cyclin-dependant kinase (CDK), or any combination thereof. Additional mRNAs include, without limitation the following having their Genbank accessions numbers in parenthesis: p75NTR (AF105292); Trk-C (XM_{—}145720); Trk-B (XM_{—}127361); NT-3 (NM_{—}008742); BDNF (AY057907); Early growth response 3 (Egr3) (NM_{—}018781); Early growth response 2 (Egr2) (XM_{—}12564); IGF-1 (NM_{—}010512); IGF-1 receptor (XM_{—}133508); IGF-2 (NM_{—}010514); IGFBP2 (NM_{—}008342); IGFBP4 (NM_{—}010517); TNF receptor superfamily, member 1a (NM_{—}011609); HGF receptor (Met) (NM_{—}008591); FGF-1 (NM_{—}010197); Fibroblast growth factor receptor 1 (Fgfr1) (NM_{—}010206); Fibroblast growth factor receptor 2 (Fgfr2) (NM_{—}010207); Fibroblast growth factor receptor 3 (Fgfr3) (NM_{—}008010); Fibroblast growth factor receptor 4 (Fgfr4) (XM_{—}193720); p27kip1 (cyclin-dependent kinase inhibitor 1B) (U09968); p19 ink4D (cyclin-dependent kinase inhibitor 2D, Cdkn2d) (NM_{—}009878); Cyclin D1 (BC044841); Cyclin E or (E1) (X75888); Cyclin G1 (NM_{—}009831); Cyclin B (X58708) (cycB mRNA for cyclin B), (NM_{—}172301) (cyclin B1 (Ccnb1), mRNA); p34 (cell cycle p34 CDC2 kinase) (AF488732); p39 or Cdk5r2 (cyclin-dependent kinase 5, regulatory subunit 2) (NM_{—}009872); p35 or Cdk5rl (cyclin-dependent kinase 5, regulatory subunit) (NM_{—}009871); CDK5 (NM_{—}007668); Ki-67 (X82786); p53 (AF151353); Bcl-2 (AH001858); Wingless-related MMTV integration site 3A (Wnt3a) (NM_{—}009522); Mus musculus wingless-related MMTV integration site 3 (Wnt3) (NM_{—}009521); Wnt14b (AB073819); Wingless-related MMTV integration site (Wnt2) (NM_{—}023653); Beta Catenin (M90364); Beta-catenin binding protein (XM_{—}127728); Glycogen synthase kinase 3 beta (GSK-3B) (NM_{—}019827); Akt1 (PKB alpha) (NM_{—}009652); Akt2 (PKB beta) (NM_{—}007434); Akt3 (PKB gamma) (NM_{—}011785) Sonic hedgehog (Shh) (NM_{—}009170); Smoothened (sonic hedgehog receptor) (AF089721); Dlx-2 (NM_{—}010054); Tubulin beta 3 (Tujl) (NM_{—}023279); CAM-L1 (NM_{—}008478); Prostaglandin-endoperoxide synthase 2 (Ptgs2 or Cox-2) (NM_{—}011198); Cerebellin 1 precursor protein (Cbln1) (XM_{—}195796); SDF1 alpha and beta (L12029) and (L12029) respectively; CXCR4 (SDF1 receptor) (NM_{—}009911); Nestin (XM_{—}130913); Aspm (AF533752); Calmbpl (NM_{—}009791); and CDC6 (cell division cycle 6 homolog) (BC052434) and (NM_{—}011799).

Step (c) can also comprise quantitatively determining the amount of proteins such as Ki-67, a cyclin, a nestin, or a cyclin-dependant kinase via detectable antibodies. Methods of protein and nucleic acid quantitative determination, mRNA extraction, antibody production and use are routine in the art, and are exemplified by the methods set forth in Sarbrook et al. (1985).

In one embodiment of the instant method, the agent has no known function. In another embodiment, the agent is a known therapeutic compound for treating a cognitive disorder (e.g. Alzheimer's, mild cognitive impairment, multi-infarctual dementia, schizophrenia or any other cognitive disorder). In a further embodiment, the agent is a known therapeutic compound for treating anxiety, depression and/or schizophrenia. In a further embodiment, the agent is a known therapeutic compound for treating a non-mental disorder. In a further embodiment, the agent is known to stimulate or inhibit a cellular pathway whose stimulation or inhibition is associated with cell division. In a further embodiment, the agent is known to bind to or otherwise effect a known receptor, transporter, enzyme or other molecular target. Agents include for example, trycyclics, serotonin reuptake inhibitors, selective norephinephrine uptake inhibitors, serotonin norepinephrine uptake inhibitors, alpha-2-adrenergic antagonists, growth factor receptor activators or modulators, phosphodiesterase inhibitors, NK1 antagonists, vasopressin V1B antagonists, mono-amino oxidase inhibitors, neuroleptics, antipsychotic inhibitors, GSK-3 beta inhibitors, and agents that upregulate the sonic hedgehog pathway. Sonic hedgehog has been shown to upregulate adult neural progenitor proliferation in vitro and in vivo (Lai et al. 2003). Agents that upregulate the sonic hedgehog pathway may be either antagonists of Patched protein or agonists of Smoothened protein in the sonic hedgehog pathway. An agent that upregulates the sonic hedgehog pathway may be either Hh-Ag 1.1, Hh-Ag 1.2, Hh-Ag 1.3 or a derivative of Hh-Ag 1.1,.Hh-Ag 1.2, Hh-Ag 1.3

This invention further provides a method for treating anxiety, depression, a cognitive disorder or a neuro-degenerative disorder by administering to an afflicted subject a therapeutically effective amount of an agent determined to have the ability to increase brain progenitor cell division, wherein such ability is determined by a method comprising (i) administering the agent to a non-human subject, and (ii) determining whether the resulting brain progenitor cell division in the subject is greater than that in a subject to which the agent was not administered.

This invention further provides a method for inhibiting the onset of anxiety, depression or a cognitive disorder by administering to a subject in need thereof a prophylactically effective amount of an agent determined as having the ability to increase brain progenitor cell division, wherein such ability is determined by a method comprising (i) administering the agent to a non-human subject, and (ii) determining whether the resulting brain progenitor cell division in the subject is greater than that in a subject Lo which the agent was not administered.

This invention further provides a method for treating anxiety, depression, a cognitive disorder or a neuro-degenerative disorder or inhibiting the onset of anxiety, depression or a cognitive disorder by administering to an afflicted subject a therapeutically effective amount of Hh-Ag 1.1, Hh-Ag 1.2, Hh-Ag 1.3, or a derivative of Hh-Ag 1.1, Hh-Ag 1.2 or Hh-Ag 1.3.

This invention further provides agents identified by the instant methods as increasing brain progenitor cell division (and anti-depressant and/or anti-anxiety activity), as well as pharmaceutical compositions comprising same (e.g. the agent and a pharmaceutically acceptable carrier), and articles of manufacture comprising a packaging material having the agent therein and a label indicating a use of the agent for treating and/or preventing the onset of anxiety, depression or a cognitive disorder in a subject.

In one embodiment, dosage is between 1 mg and 200 mg per human subject, or weight equivalent thereof for a non-human subject. In a further embodiment, dosage is between 5 mg and 50 mg per human subject, or weight equivalent thereof for a non-human subject. In a further embodiment, dosage is between 10 mg and 20 mg per human subject, or weight equivalent thereof for a non-human subject.

Further envisioned is a method for identifying agents for the treatment of anxiety, depression, cognitive impairment, Alzheimer's disease, Parkinson's disease and stroke based on their ability to increase the levels of nucleic acid or protein markers recited in claim 8. Still further envisioned is a method for identifying drug targets for the treatment of anxiety, depression, cognitive impairment, Alzheimer's disease, Parkinson's disease and stroke based on the ability of agents that interact with the drug target to increase the level of nucleic acid or protein marker levels recited in claim 8. Still further envisioned is a method for determining the onset of action of an agent by measuring the increase in nucleic acid or protein markers recited in claim 8 after multiple durations of agent administration. Still further envisioned is a method for determining the efficacy of an agent by measuring the increase in nucleic acid or protein marker levels recited in claim 8 following administration of different doses of the agent to estimate the maximal increase. Still further envisioned is a method for determining the duration of action of an agent by measuring the increase in nucleic acid or protein marker levels recited in claim 8 at multiple times after cessation of agent administration.

This invention is illustrated in the Experimental Details section which follows. This section is set forth to aid in an understanding of the invention but is not intended to, and should not be construed to, limit in any way the invention as set forth in the claims which follow thereafter.

Experimental Details

Synopsis

The fact that antidepressant medications display delayed therapeutic efficacy suggests that their action results from slowly developing changes in the brain rather than from their immediate pharmacological effects. Various chronic antidepressant treatments have been found to increase neurogenesis in the dentate gyrus of adult animals, but the functional significance of this phenomenon has not been demonstrated.

This invention is based on work using genetic and radiological methods to show that disrupting anti-depressant-induced neurogenesis blocks behavioral responses to antidepressants. Specifically, this work shows that %-HT1A receptor null mice are insensitive to the neurogenic and behavioral effects of selective serotonin reuptake inhibitors (SSRIs; e.g., fluoxetine). Further, X-irradiation of a restricted region of the mouse brain containing the hippocampus prevents the neurogenic and behavioral effects of two classes of antidepressants. These findings indicate that the behavioral effects of chronic antidepressants can be mediated by the ability of these drugs to stimulate neurogenesis in the hippocampus.

Materials and Methods

The present study determined whether an increase in neurogenesis is required for the efficacy of ADs. To test this hypothesis, this study: (1) used mice with a genetic deletion that disrupts SSRI-induced neurogenesis; and (2) developed a radiological procedure to block neurogenesis specifically in the hippocampus. The consequences of these manipulations were assessed in two behavioral paradigms that detect chronic antidepressant-like responses.

Animals

Knockout and wild-type 129/SvEv age-matched adult male mice (12-20 weeks) derived from heterozygote crossings (Phillips et al., 1999) were used in all experiments that included 5-HT_{1A} knockout mice. For all the other experiments except the CUS paradigm, 129/SvEv mice of the same sex and age were purchased from Taconic (Germantown, N.Y.). BALB/c mice were used in the CUS paradigm due to their ability to react to chronic stress (Kopp et al., 1999). Mice were housed 4-5 per cage in a 12-h (06:00-18:00) light-dark colony room at 22° C. with freely available food and water.

Behavioral Testing and Chronic Unpredictable Stress Procedure

The Novelty-suppressed feeding test was carried out during a 5-min period as previously described (Santarelli et al., 2001). Briefly, the testing apparatus consisted of a plastic box, 50×50×20 cm. The floor was covered with approximately 2 cm of wooden bedding. Twenty-four hours prior to behavioral testing, all food was removed from the home cage. At the time of testing, a single pellet of food (regular chow) was placed on a white paper platform positioned in the center of the box. An animal was placed in a corner of the maze and a stopwatch was immediately started. The measure of interest (chewing) was scored when the mouse was sitting on its haunches and biting with the use of forepaws. Immediately after this test, mice were transferred to their home cage and the amount of food consumed in 5 min was measured (home cage food consumption). In knockout experiments, mice were genotyped after behavioral testing to remove the possibility of investigator bias. Mice were always tested during the light period.

The stress regimen used is a variant of the chronic mild stress procedures described by Willner et al. (Willner et al., 1992). Mice were subjected to various and repeated unpredictable stressors for a period of five weeks (Griebel et al., 2002). The different stressors used were: alterations of the bedding (repeated sawdust changing, removal of sawdust, damp sawdust, substitution of sawdust with 37° C. water); cage tilting (45°); predator sounds (15 min.); cage shift (mice were positioned in the empty cage of another male) ; light to dark or dark to light shifts (four light/dark successions of 30 min every 24 hours); reversal of the light/dark cycle. The fluoxetine treatment (10 mg/kg/day, i.p.) started at the beginning of the third week. The state of the coat was assessed at the end of the stress regimen (end of fifth week). The total score resulted from the sum of the score of seven different body parts: head, neck, dorsal coat, ventral coat, tail, forepaws and the hindpaws. A score of 0 was given for a well-groomed coat and 1 for a spoiled coat (see Griebel, 2002). The grooming latency was assessed at the end of the sixth week. This test consisted in squirting 200 μl of a 10% sucrose solution on the mouse's snout. The latency to start grooming was then recorded.

Cued Fear Conditioning

Fear conditioning took place in a mouse conditioning chamber (model ENV-010MD; Med Associates, St. Albans, Vt.) enclosed by a sound-attenuating cubicle (model ENV-018M, Med Associates). Stimulus presentation was controlled by a programmable audio generator (model ANL-926, Med Associates). On the training day mice were placed in the conditioning chamber and, following a two-minute habituation period, presented with three acoustic tone CSs (4.5 kHz, 20 sec, 85 dB) that co-terminated with a footshock US (0.7 mA, 1 sec). The CS/US pairings occurred 120, 290 and 400 seconds after placing the animals in the chamber. 24 hours later, mice were placed in a novel conditioning chamber containing different spatial and sensory cues, and presented with the CS after 90 seconds. Freezing (characteristic crouching posture, and cessation of all but respiration-related motion) was used as an index of conditioned fear (Blanchard and Blanchard, 1969; Blanchard and Blanchard, 1969; LeDoux et al., 1990; LeDoux et al., 1984) and scored as the percentage of time spent freezing for 20 seconds preceding and 20 seconds during the CS.

Drugs

Fluoxetine (Eli Lilly, Indianapolis, Ind.), Imipramine (Sigma-Aldrich, St. Louis, Mo.), Desipramine (Sigma-Aldrich, St. Louis, Mo.) and Haloperidol (Sigma-Aldrich, St. Louis, Mo.) were dissolved in tap water at a concentration of 80, 160, 160, and 8 mg/L, respectively. These concentration were established based on the average drinking amount and average weight of the mice used in the study to achieve a final dose of 10 (fluoxetine), 20 (imipramine and desipramine) and 1 (haloperidol) mg/kg/day. HPLC analysis demonstrated that steady serum levels of fluoxetine, nor- fluoxetine, imipramine and desipramine had been achieved by 5 days after the beginning of the oral regimen (data not shown). Vehicle animals received tap water. 8-hydroxy-2-(di-n-propylamino) tetralin (8-OH-DPAT, Sigma-Aldrich, St. Louis, Mo.) was dissolved in sterile saline (0.9% NaCl) at a concentration of 4.5 mg/ml, and delivered through Alzet osmotic mini-pumps (model 2004, Alzet, Cupertino, Calif.) implanted subcutaneously 28 days prior to behavioral testing. The final average dose delivered was 1 mg/kg/day. Sham mice were implanted with mini-pumps containing sterile saline.

BrdU Labeling, Immunohistochemistry and Imaging

To assess the effect of antidepressant or X-irradiation treatments on the number of BrdU-positive cells, mice were administered BrdU (4×75 mg/kg i.p., dissolved in saline, every 2 hr; Roche Diagnostic, Indianapolis, Ind.) and sacrificed 24 hr after the last BrdU injection (FIG. 2A, 3B, 5C-D). To determine cell survival and phenotype after the various antidepressant treatments, fluoxetine, imipramine and vehicle-treated mice were allowed to survive for 28 days after the last BrdU injection (FIG. 2C-D, 3C). After deep anesthesia with a mixture of ketamine/xylazine (100 mg/kg and 7 mg/kg respectively),, mice were transcardially perfused (cold saline for 2 min, followed by 4% cold paraformaldehyde/0.1 M phosphate buffer for 5 min) and brains were collected for immunohistochemistry. All brains were post-fixed overnight in 4% paraformaldehyde at 4° C., then cryoprotected in 30% sucrose and stored at 4° C. Serial sections of the brains were cut (35 μm sections) through the entire hippocampus (plates 41-61; Franklin and Paxinos, 1997) on a cryostat and stored in PBS with 0.1% NaN₃. For DAB staining, sections were slide-mounted and boiled in citric acid, pH 6.0, for 5 min, rinsed with PBS, and treated with 0.01% trypsin in Tris/CaC12 for 10 min. Brain sections were incubated for 30 min with 2N HCl and blocked with 5% NGS (Jackson ImmunoResearch, West Grove, Pa.). Sections were then incubated over night at room temperature with anti-mouse BrdU (1:100; Becton-Dickinson). After washing with PBS, sections were incubated for 1 hr with secondary antibody (1:200 biotinylated goat anti-mouse; Sigma-Aldrich, St. Louis, Mo.) followed by amplification with an avidin-biotin complex (Vector Laboratories). The staining was visualized with DAB (Sigma-Aldrich, St. Louis, Mo.). For fluorescent double-labeling, the DNA-denaturation procedure consisted of the following steps: 2 hr incubation in 50% formamide-2×SSC at 65° C., 5 min rinse in 2×SSC, 30 min incubation in 2N HCl at 37° C., and 10 min rinse in 0.1 M boric acid, pH 8.5. Sections were then incubated in TBS-0.1% Triton X-100 (Tx)-5% normal donkey serum (NDS) for 30 min, followed by anti-rat BrdU primary antibody (1:100; Accurate Westbury, N.Y. or Serotec, Oxford, UK) in TBS/NDS/Tx for 24 hrs at 4° C. in combination with either a mouse monoclonal antibody to NeuN (1:500; Chemicon, Temecula, Calif.) or a rabbit polyclonal antibody to GFAP (1:800; DAKO, Carpinteria, Calif.). The fluorescent secondary antibodies used were biotin-conjugated donkey anti-rat (1:200) plus streptavidin Cy2 (1:200), donkey anti-mouse. Cy3 (1:500), or donkey anti-rabbit Cy3 (1:500) (Jackson ImmunoResearch, West Grove, Pa.). Fluorescent images were taken with a Bio-Rad MRC 1000 confocal laser scanning system coupled to a Zeiss Axiovert 100 inverted microscope. For each image acquisition, a Kalman average of five frames was used. For quantification of BrdU/NeuN or BrdU/GFAP double-labeled cells, at least 50 BrdU-positive cells per animal were analyzed using Z-plane sectioning (1 μm steps).

Irradiation

Mice were anesthetized with ketamine and xylazine, placed in a stereotaxic frame (Kopf, Tujunga, Calif.) and exposed to cranial irradiation using a Siemens Stabilopan X-ray system (Hamburg, Germany) operated at 300 kVp and 20 mA. Animals were protected with a lead shield that covered the entire body, but left unshielded a 3.22×11-mm treatment field above the hippocampus (interaural 3.22-0.00). Dosimetry was done using a Capintec Model PR06G electrometer ionization chamber. (Capintec, Ramsey, N.J.) and Kodak Readypack Radiographic XV films (Kodak, Rochester, N.Y.). The corrected dose rate was approximately 1.8 Gy per min at a source to skin distance of 30 cm. The procedure lasted 2 min and 47 sec, delivering a total of 5 Gy.

Stereology

Morphological analyses to assess the effects of X-irradiation on Nissl-stained granule cells were performed as previously described (Liberatore et al., 1999). Briefly, the total number of Nissl-stained cells from 6 sham and 6 X-irradiated mice was counted monolaterally in the dentate gyrus, through its entire rostro-caudal axis, by using an unbiased method of cell counting (optical fractionator; (Liberatore et al., 1999). Throughout the experiment, the investigator was blind to the treatment status. For this analysis, a Zeiss Axioplan-2 microscope equipped with a Zeiss planapochromat 100× oil objective and a CCD camera is used to generate digitized images that will be collected and analyzed on a Micron Millennia computer using the software Stereo Investigator™ (MicrbBrightField Inc., Colchester, Vt.). Every sixth section throughout the entire extent of the dentate gyrus was counted. The volume was then calculated, taking into account the frequency of sections (1:6) and their thickness (35 μm), according to the Cavalieri principle (Liberatore et al., 1999).

Corticosterone Test

To establish baseline levels of corticosterone, blood samples were collected by making a small incision on the tip of the mouse tail. Twenty four hours later the same animals were subjected to an Open Field (Ramboz et al., 1998), and 15 min later animals were scarified and trunk blood was drawn. Serum corticosterone concentrations were measured with an ¹²⁵I-RIA kit (ICN, Costa Mesa, Calif.).

Electrophysiological Recordings

Hippocampal slices were obtained from sham and irradiated mice four weeks after the beginning of the X-ray procedure. Transverse slices (thickness 400 μm), prepared as previously described (Vitolo et al., 2002), were maintained in an interface chamber at 29° C. and perfused with ACSF solution (124.0 mM NaCl, 4.4 mM KCl, 1.0 mM Na2HPO4, 25.0 mM NaHCO3, 2.0 mM CaCl2, 2.0 mM MgSO4, 10 mM glucose) continuously bubbled with 95% O₂ and 5% CO₂. Slices were with ACSF solution. Both the stimulating and the recording electrodes were placed in CA1 stratum radiatum (SR). Basal synaptic transmission was assayed by plotting the stimulus voltages (V) against slopes of fEPSP to generate input-output relationships. For LTP experiments, baseline stimulation was delivered every minute (0.01 ms pulse duration) at an intensity corresponding to approximately 35% of that used for the maximum evoked response. Baseline responses were recorded for 15 min prior to the LTP-inducing stimulation to assure stability of the response. LTP was induced using theta-burst stimulation (4 pulses at 100 Hz, with the bursts repeated at 5 Hz and each tetanus including 3 ten-burst trains separated by 15 seconds).

Results

The Novelty Suppressed Feeding Paradigin Detects Chronic Antidepressant Actions in Mice

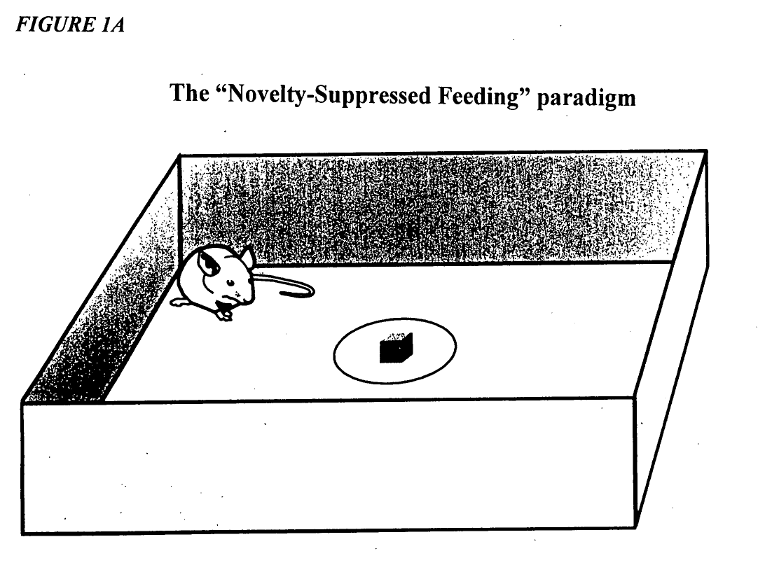

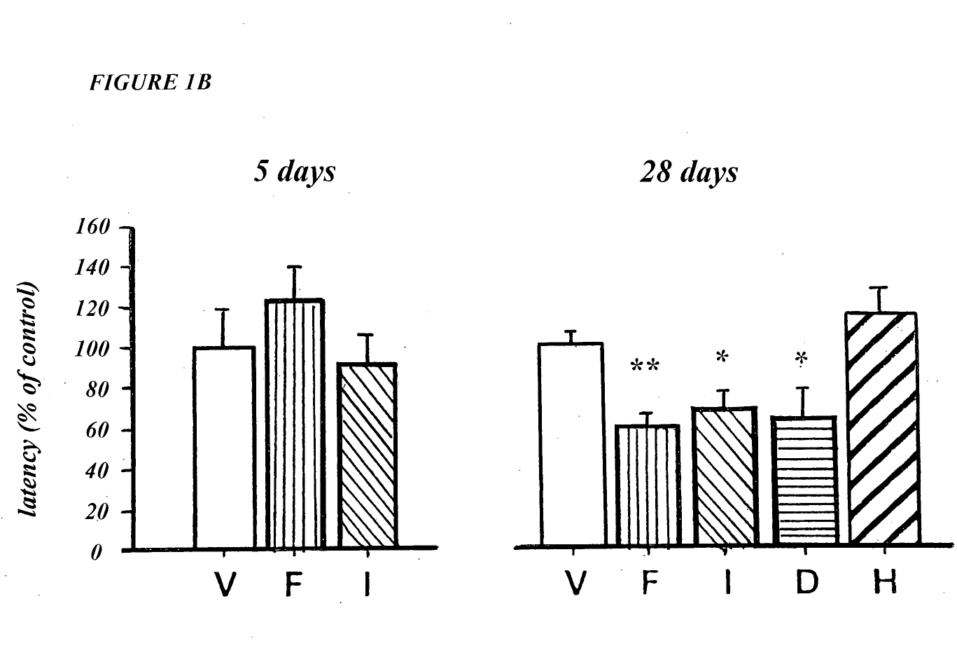

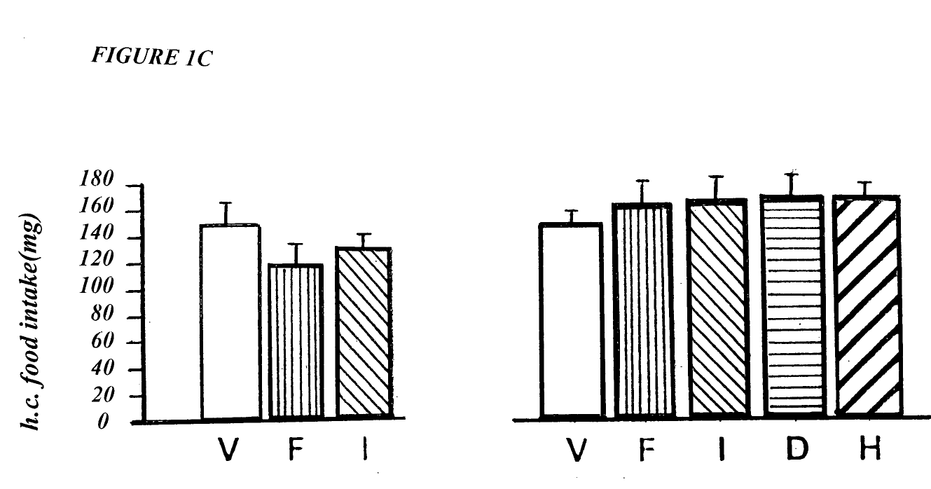

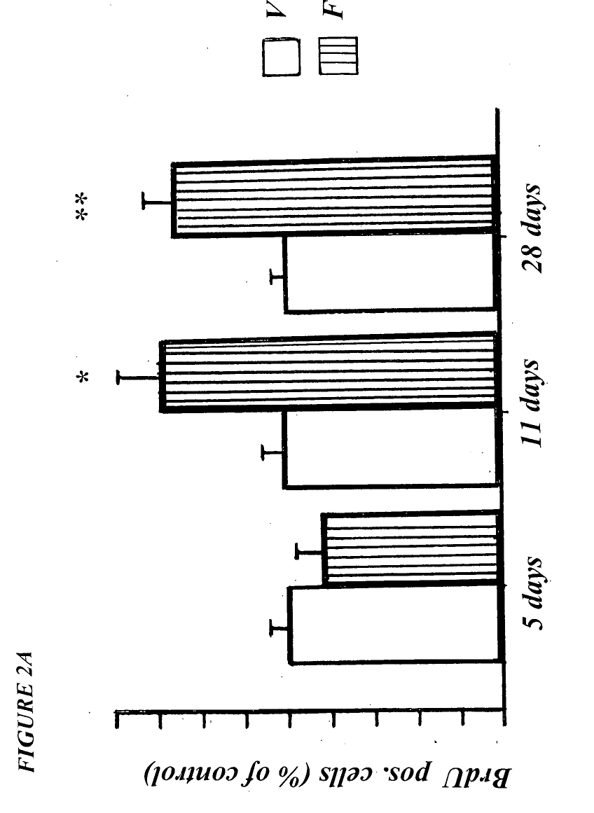

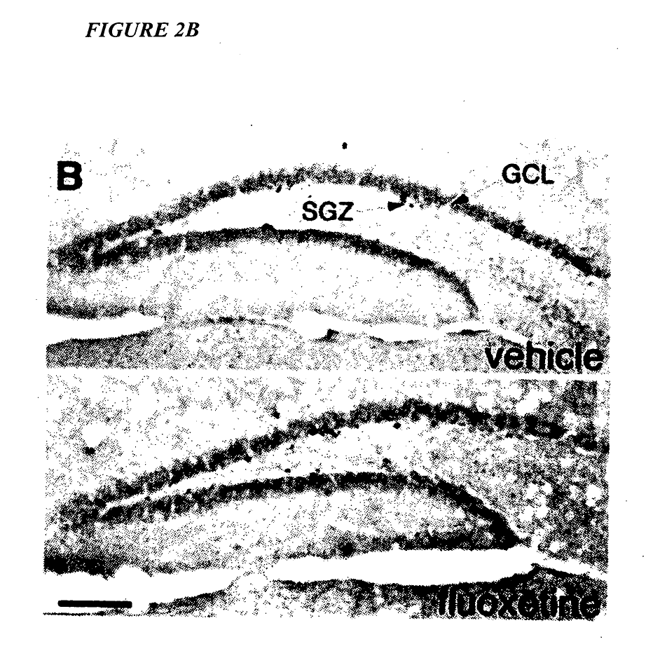

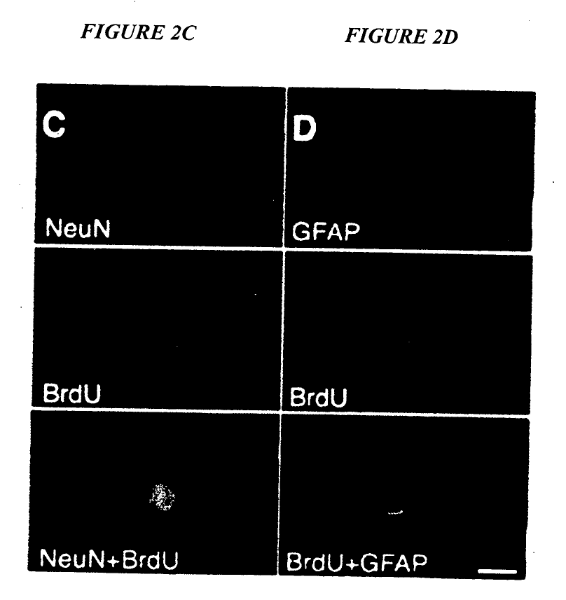

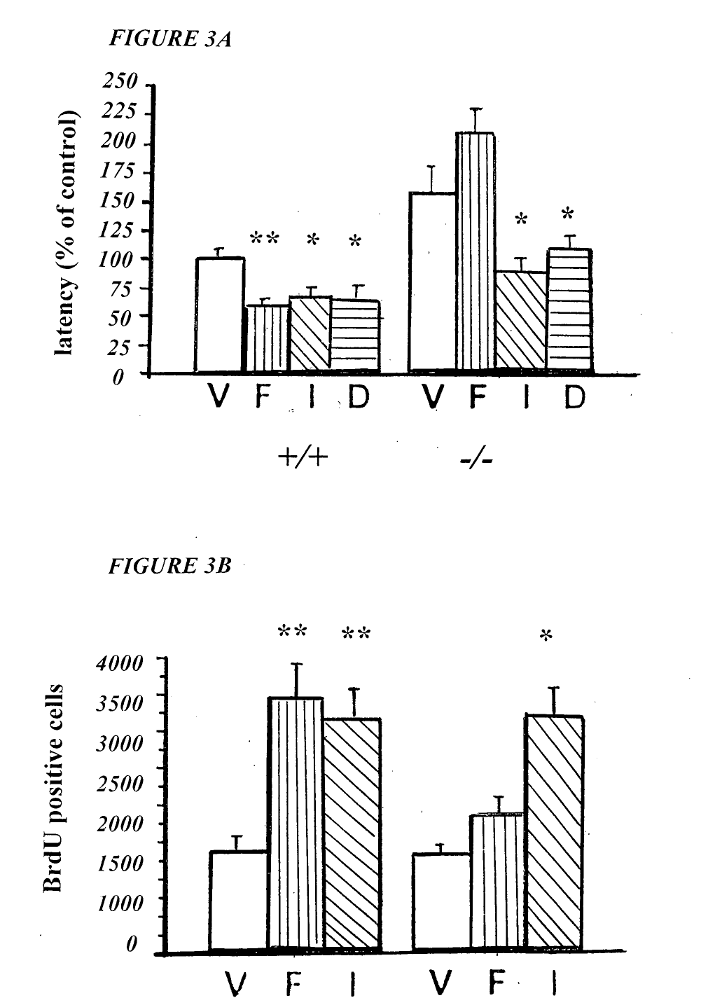

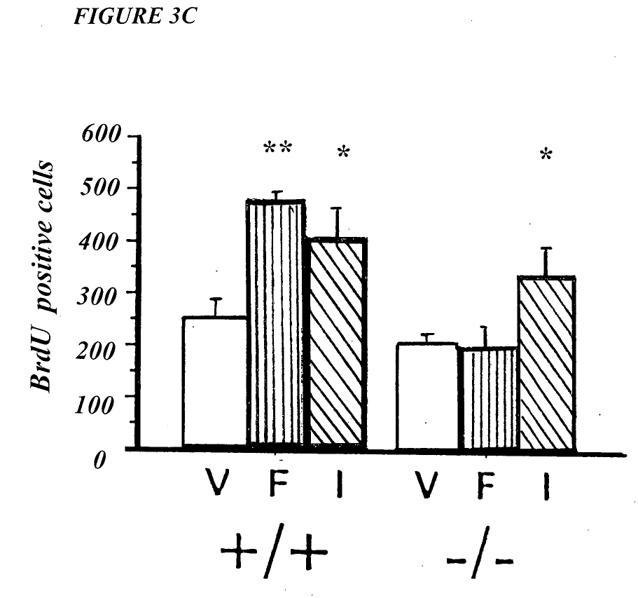

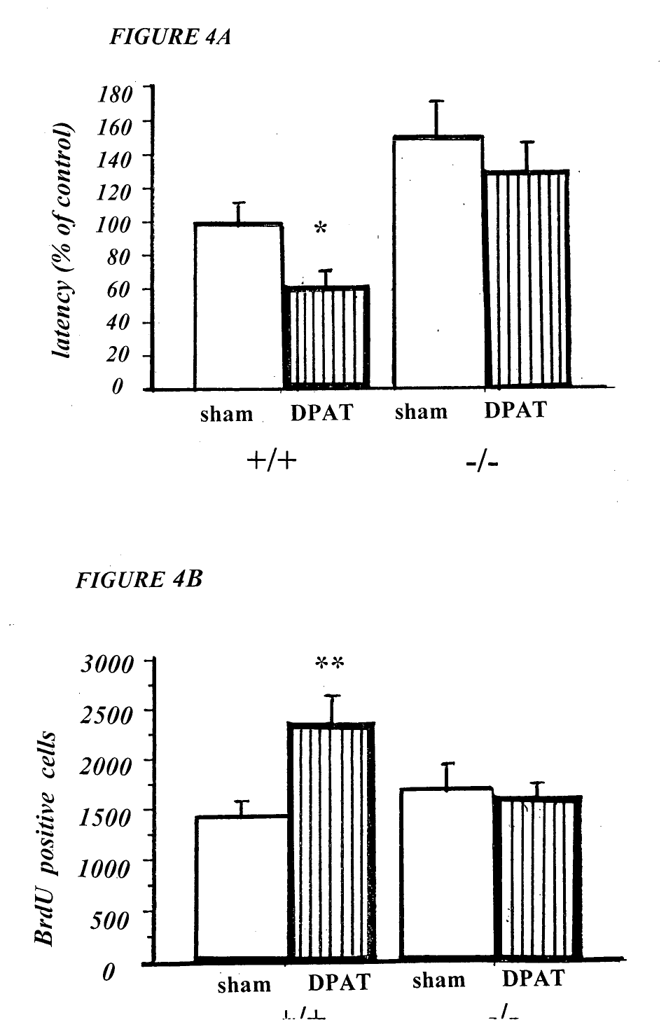

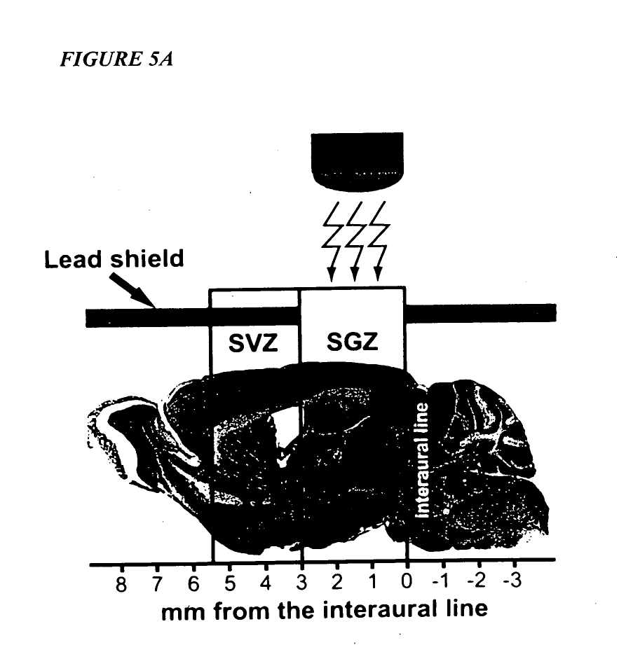

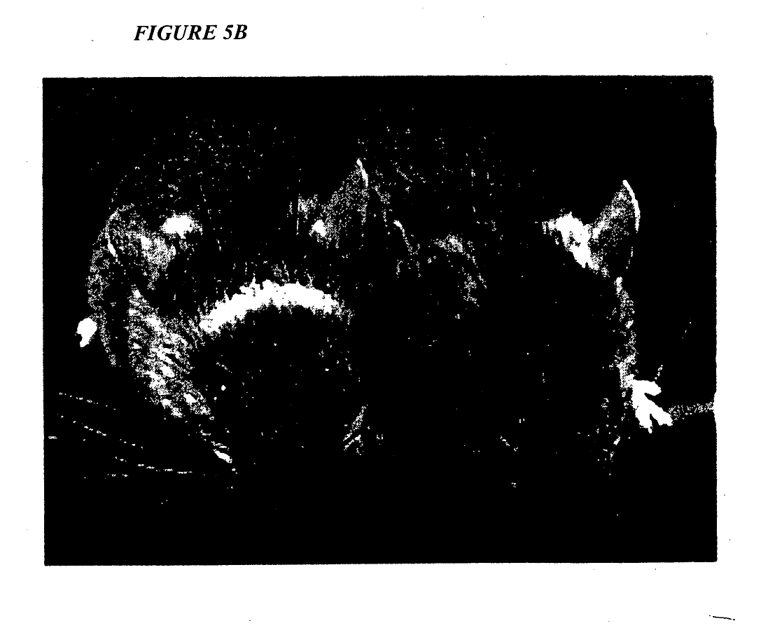

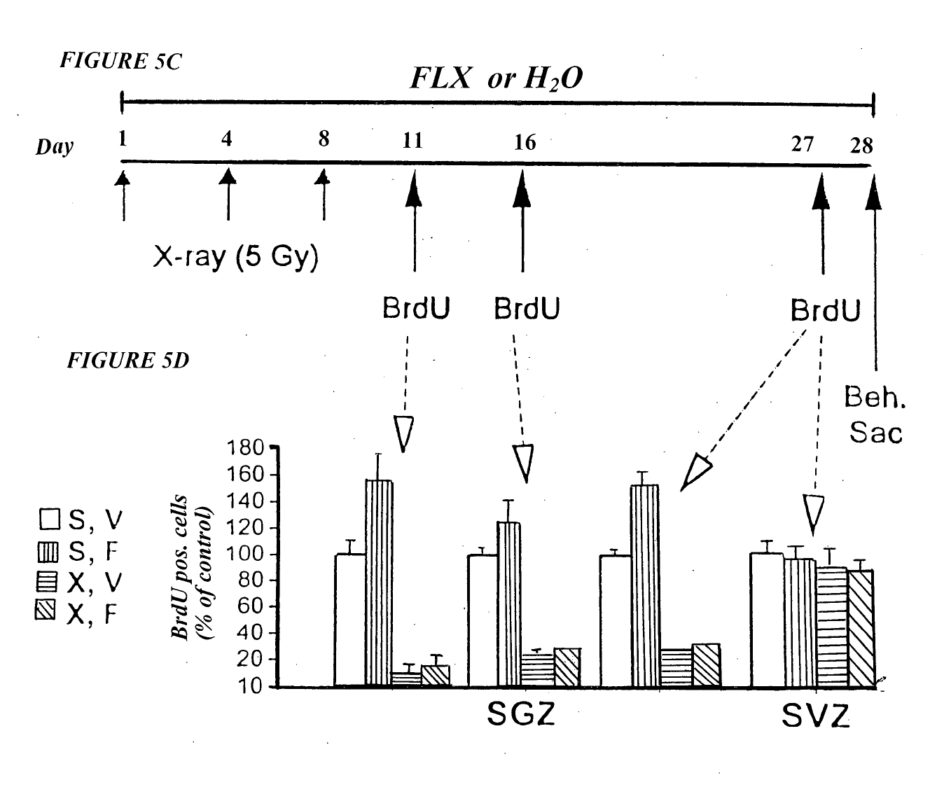

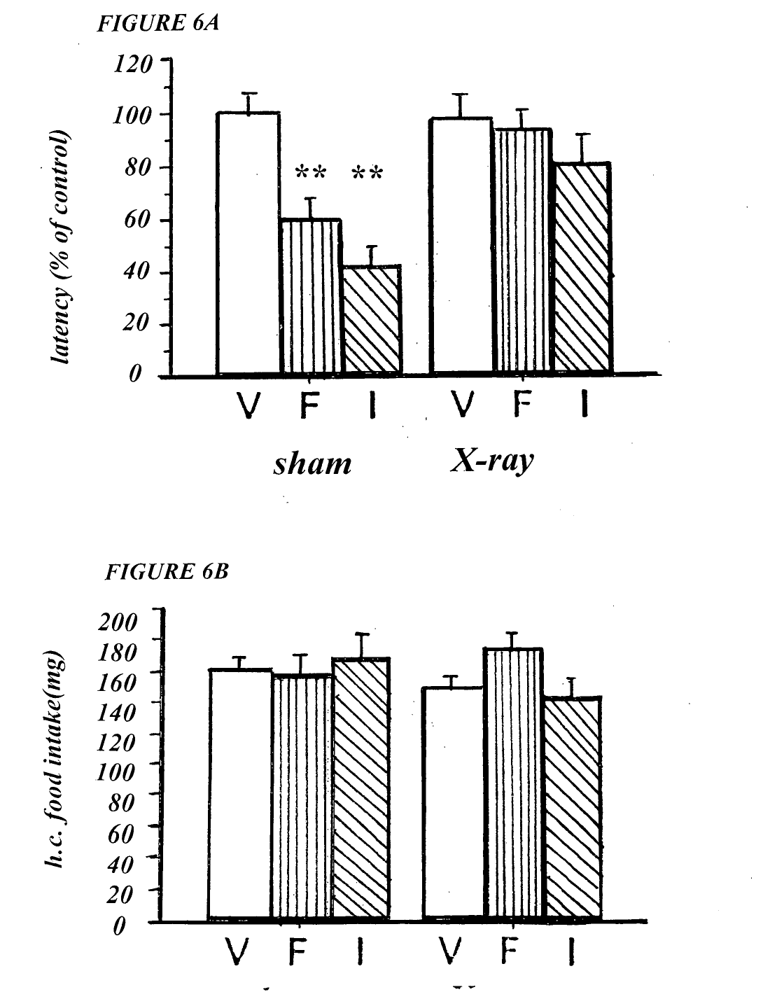

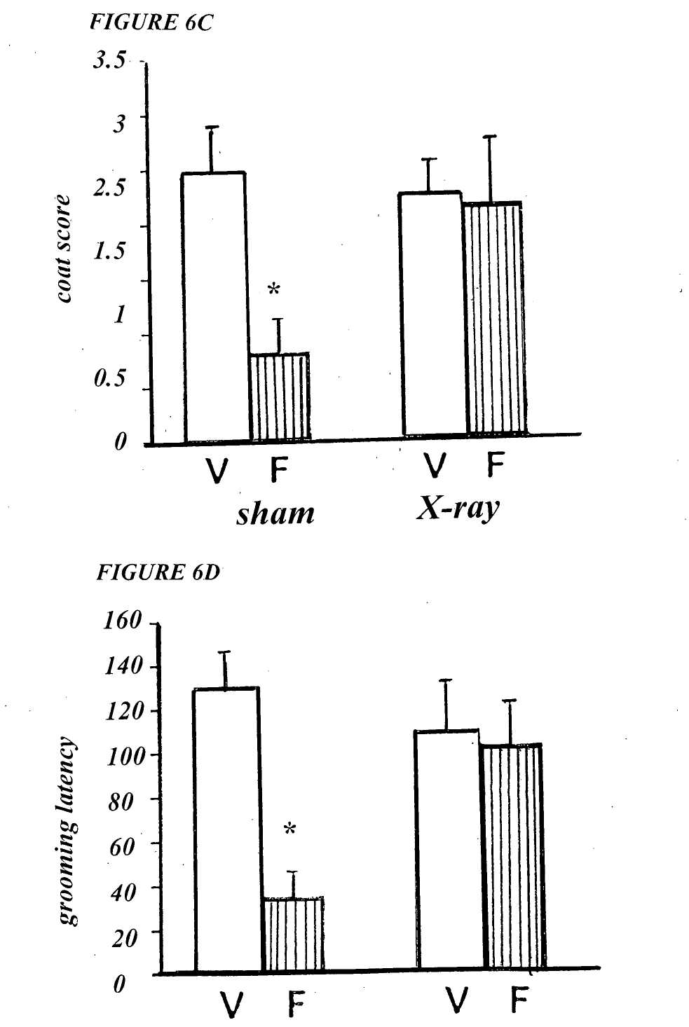

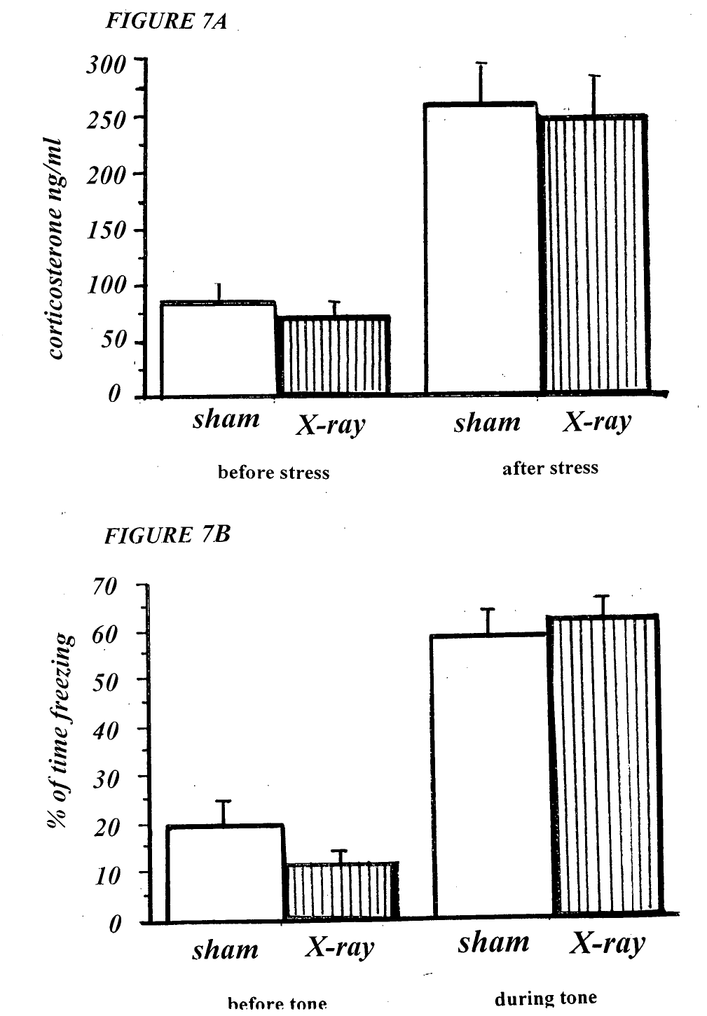

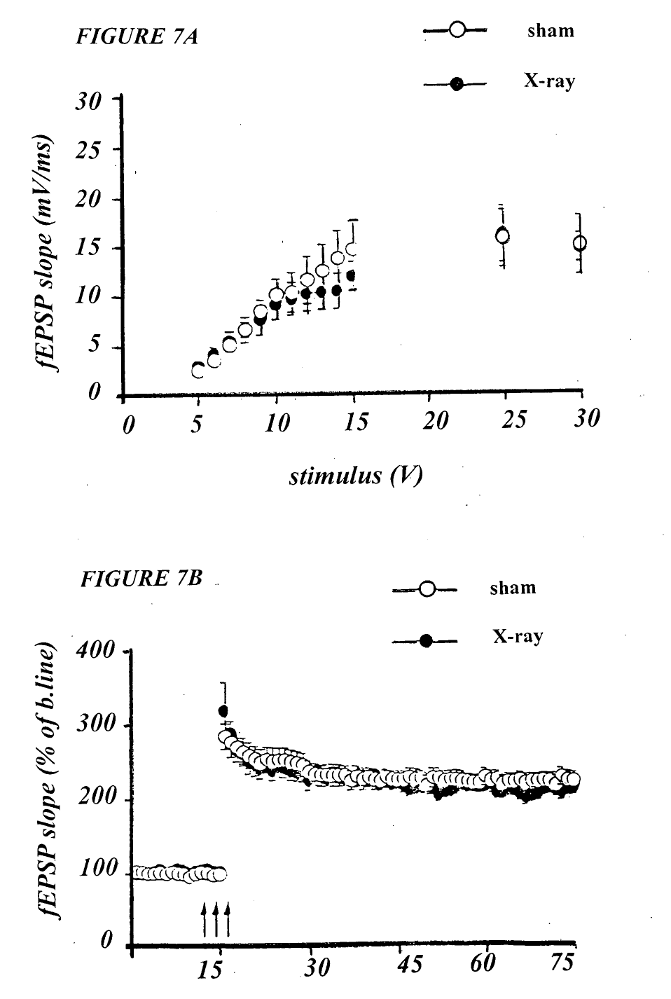

Few behavioral paradigms have been able to reliably demonstrate changes in mouse behavior after chronic, but not acute, treatment with antidepressant drugs (Cryan et al., 2002), We have adapted the novelty-suppressed feeding test (NSF), which has been used to assess chronic antidepressant efficacy in rats (Bodnoff et al., 1988; Bodnoff et al., 1989), to the 129/Sv mouse strain used in this study. The NSF is a conflict test in which food-deprived animals are presented with a food pellet placed in the center of a brightly-lit open field. This paradigm elicits competing motivations: the drive to eat the food pellet, and the fear of venturing into the center of the arena. The dependent variable in this test is the amount of time it takes for an animal to begin eating the pellet, and this latency has been used as an index of anxiety-like behavior because it is decreased by classical anxiolytic drugs such as benzodiazepines (Bodnoff et al., 1988; Bodnoff et al., 1989; Shephard and Broadhurst, 1982) (FIG. 1A).

To determine whether ADs have an effect in this test, we treated adult mice with fluoxetine (10 mg/kg/day), imipramine (20 mg/kg/day), desipramine (20 mg/kg/day), haloperidol (1 mg/kg/day), or vehicle, delivered via the drinking water. We chose ADs that act through different mechanisms: fluoxetine, a selective serotonin reuptake inhibitor; imipramine, a tricyclic antidepressant that blocks the reuptake of both 5-HT and NE; and desipramine, a tricyclic antidepressant that selectively blocks NE reuptake. Haloperidol, an antipsychotic that antagonizes D2 dopamine receptors, was used as a negative control because it is devoid of antidepressant activity in humans. NSF performance was assessed after either 5 or 28 days of oral drug administration. As shown in FIG. 1B, a 5-day treatment with either fluoxetine or imipramine had no effect on the animals' latency to feed as compared to vehicle-treated animals. In contrast, all three antidepressants, but not haloperidol, produced significant decreases in the latency to feed in animals treated for 28 days. These results suggest that, as in rats, the NSF paradigm reliably detects behavioral effects of chronic antidepressant administration in mice.

Antidepressants are known to have various effects on appetite. To control for this potential confounding factor, the feeding drive of each animal was assessed by returning it to the familiar environment of the home cage immediately after the test, and measuring the amount of food consumed over a period of 5 minutes. FIG. 1C shows that none of the drugs tested produced a significant change in the animals' food consumption after either sub-chronic or chronic treatment. There was a trend for both fluoxetine and imipramine to slightly decrease food intake after a 5-day administration, which is consistent with the anorectic effect these drugs may produce in humans at the beginning of an antidepressant treatment (Masand and Gupta, 1999). These results demonstrate that chronic treatment with Ads that target distinct monoamine systems can produce significant changes in the behavior of mice in the NSF test. Moreover, the slow appearance of these changes resembles the delay in the onset of ADs efficacy in humans, and further validates the NSF paradigm as an animal model of chronic antidepressant action.

Chronic Fluoxetine Increases Neurogenesis in the Dentate Gyrus

It has recently been shown that various antidepressant treatments, including fluoxetine, increase neurogenesis in the dentate gyrus (DG) of the rat hippocampus (Malberg et al., 2000). These drugs induce an increase in the number of progenitor cells that incorporate the DNA synthesis marker BrdU, but do not alter the proportion of these cells that survive and differentiate into mature neurons (Malberg et al., 2000). To confirm that chronic fluoxetine induces changes in the number of BrdU-positive cells in the DG, we treated adult mice with either vehicle or fluoxetine for 5, 11 or 28 days. On the final day of treatment, all animals were injected with BrdU (four injections of 75 mg/kg), sacrificed 24 hours later, and perfused for immunohistochemistry. The, treatment with fluoxetine caused a 60% increase in the number of BrdU positive cells in the DG of animals after either 11 or 28 days, but had no effect in animals treated for 5 days (FIGS. 2A and B).

Several studies have now demonstrated that cells born in the subgranular zone (SGZ) of the adult rodent hippocampus express markers of adult neurons as they differentiate and mature (Gould and Gross, 2002). To determine the fate of hippocampal cells labeled with BrdU following chronic treatment with either fluoxetine or vehicle, animals were allowed to survive for a period of four weeks after injection of BrdU. During this time fluoxetine oral treatment was suspended and animals were returned to tap water. Brain sections were co-labeled with antibodies raised against BrdU and either the mature neuronal marker NeuN or the astroglial marker GFAP. FIG. 2C shows an example of BrdU-positive cells co-labeled with NeuN or GFAP. The percentage of BrdU-positive cells within the SGZ and granule cell layer that expressed NeuN was 70±2%, and the percentage of BrdU-positive cells that expressed GFAP was 15±3%. These proportions were not influenced by the antidepressant treatment, as previously reported (Malberg et al., 2000). These data show that chronic treatment with fluoxetine results in an increased number of cells generated in the DG, the majority of which acquire a mature neuronal phenotype, in agreement with previous reports (Feng et al., 2001; Kempermann et al., 1997; Malberg et al., 2000).

In summary, these findings show that fluoxetine starts to augment cell division in the DG between 5 and 11 days after treatment begins, and that this effect leads to a subsequent increase in the number of cells that exhibit a mature neuronal phenotype by day 28 (see also FIG. 3C). Moreover, the time required for the generation of new cells and their differentiation into mature neurons parallels the delay in the appearance of behavioral effects induced by ADs treatment in the NSF test.

The 5-HT_{1A} Receptor is Required for the Behavioral Effects of Fluoxetine in the NSF Test

Among the 14 known 5-HT receptor subtypes, the 5-HT_{1A} receptor has been most consistently implicated in the modulation of mood and anxiety-related behaviors (Gross et al., 2000; Sargent et al., 2000). To determine whether this receptor is required to mediate the effects of serotonin- and noradrenaline-enhancing ADs, we compared the effects of these drugs in wild type (WT) mice and in mice lacking the 5-HT_{1A} receptor (5-HT_{1A} receptor knockout (KO) mice). WT and KO mice were treated with fluoxetine, imipramine, desipramine, or vehicle for a period of 28 days before being tested in the NSF paradigm. FIG. 3A shows that mice lacking the 5-HT_{1A} receptor display a higher latency to eat the food pellet than their littermate controls, in agreement with their increased levels of anxiety-like behaviors reported previously (Gross et al., 2000; Gross et al., 2002). In addition, we found that the KO mice were insensitive to the effects of chronic fluoxetine, but were still responsive to both imipramine and desipramine (FIG. 3A).

To further investigate the role of 5-HT_{1A} receptors in mediating antidepressant-like responses in the NSF test, we assessed the effect of a direct pharmacological activation of these receptors. We administered the selective 5-HT_{1A} receptor agonist 8-OH-DPAT subcutaneously for 28 days via osmotic mini pumps, and tested the animals in the NSF paradigm at the end of the treatment. As shown in FIG. 4A, 8-OH-DPAT significantly decreased the latency to feed in the WT mice, but was ineffective in 5-HT_{1A} receptor KO animals, indicating that the effect of 8-OH-DPAT is mediated by 5-HT_{1A} receptors.

These data show that activation of the 5-HT_{1A} receptor is sufficient to produce antidepressant-like effects in the NSF test. In addition, the results suggest that the 5-HT_{1A} receptor mediates, at least in part, the effect of fluoxetine in this test, but is not necessary for similar behavioral effects produced by drugs targeting the noradrenergic system.

The 5-HT_{1A} Receptor is Required for the Effects of Fluoxetine on Hippocampal Neurogenesis

Adult hippocampal neurogenesis has recently emerged as a form of plasticity that may underlie some of the functional changes required for the therapeutic effects of antidepressants (Chen et al., 2000; Malberg et al., 2000; Manev et al., 2001). Since 5-HT_{1A} receptor KO mice are not responsive to the behavioral effects of serotonin selective ADs in the NSF, but still respond to ADs acting through a noradrenergic mechanism, they represent an excellent tool to investigate potential correlations between the behavioral effects of ADs and their effects on hippocampal neurogenesis.

In this set of experiments, wild type and 5-HT_{1A} receptor KO mice were injected with BrdU following 27 days of treatment with fluoxetine, imipramine, or vehicle. To assess the effect of AD treatment on cell proliferation, one group of mice was sacrificed 24 hours after BrdU injection and sections containing the dentate gyrus were analyzed by immunohistochemistry. Strikingly, while fluoxetine caused a two-fold increase in BrdU-labeled cells in wild type mice, it had no effect in 5-HT_{1A} receptor KO mice (FIG. 3B). Further paralleling the behavioral data (FIG. 3A), chronic treatment with imipramine induced a significant increase in BrdU-labeled cells in both wild type and knockout mice (FIG. 3B). To determine whether chronic antidepressant treatment affects the survival of newborn cells, a second group of animals was sacrificed 28 days after BrdU injection. The results show that, irrespective of genotype and treatment, approximately 20% of the BrdU-positive cells observed 24 hours after injection survive for 28 days (FIG. 3C), in agreement with previous reports using the 129/Sv strain (Kempermann et al., 1997). The pattern of responsiveness to the drugs tested remained similar to that observed in the proliferation experiment (FIG. 3B); specifically, fluoxetine had an effect in WT but not in KO, while imipramine was effective in both genotypes (FIG. 3C).

These results indicate that 5-HT_{1A} receptors are required for fluoxetine, but not imipramine-induced neurogenesis. To test whether activation of 5-HT_{1A} receptors is sufficient to elicit an increase in neurogenesis, WT and 5-HT_{1A} receptor KO mice were treated chronically with 8-OH-DPAT or vehicle before injection with BrdU. In WT mice, chronic treatment with 8-OH-DPAT caused an increase in cell proliferation similar to that seen after treatment with fluoxetine or imipramine (FIG. 4B). This effect was not observed in KO mice, indicating that the action of 8-OH-DPAT was specific to 5-HT_{1A} receptors.

In conclusion, these experiments demonstrate that 5-HT_{1A} receptors are necessary and sufficient to mediate both the behavioral and neurogenic effects of SSRIs, but are not required to mediate the effects of noradrenergic-enhancing ADs. These data also reveal a tight correlation between increases in neurogenesis elicited by fluoxetine or 8-OH-DPAT and the behavioral efficacy of these drugs and, thus, suggest a role for hippocampal neurogenesis in mediating the action of ADs.

Irradiation Suppresses Adult Hippocampal Neurogenesis

In order to explore the possibility that hippocampal neurogenesis participates in the mechanism of action of ADs, we tested whether the blockade of neurogenesis would mitigate the behavioral effects of these drugs. Recent studies have shown that antimitotic drugs delivered either peripherally or via direct infusion onto the brain surface can block cell division in the proliferative areas of the adult rodent brain (Doetsch et al., 1999; Seri et al., 2001; Shors et al., 2001). In one of these studies, the systemic administration of the anti-mitotic agent MAM caused a dramatic reduction in neurogenesis, as well as learning deficits in a hippocampal-dependent task (Shors et al., 2001). However, the exposure to MAM was not restricted to the hippocampus and likely disrupted cell proliferation in other brain structures, such as the subventricular zone, as well as in the periphery. To avoid such confounding effects, we devised a procedure to selectively block neurogenesis in the hippocampus.

Long-term reductions in cell proliferation within the DG have previously been reported following low dose X-irradiation of the heads of rats (Tada et al., 2000). To determine whether focal irradiation can produce similar effects in mice, we delivered fractionated, low doses of X-rays to the hippocampus, while sparing the body and majority of the brain. To accomplish this, we positioned anesthetized mice in a stereotaxic frame and covered the entire body and head with protective lead shielding. An opening was created in the shield directly above the hippocampus. This window was constructed based on stereotaxic coordinates and positioning was confirmed surgically (FIG. 5A and B, also see methods). The amount of irradiation was titrated to the minimum dose necessary to produce a significant and long-lasting reduction of cell proliferation in the dentate gyrus (FIG. 5D). Three 5-Gray doses of X-ray were delivered from a distance of 30 cm above the top of the head. Adult mice were irradiated on day 1,4, and 8, and quantitative analysis of cell proliferation in the SGZ and SVZ was subsequently conducted by BrdU labeling. To assess the effects of this procedure at different time points, irradiated and sham mice were injected with BrdU on day 11, 15 or 27 and all animals were sacrificed on day 28 (FIG. 5C). As shown in FIG. 5D, irradiation resulted in ˜85% reduction in BrdU-positive cells in the SGZ. This effect persisted at the time of behavioral testing (FIG. 5C,D) and lasted for at least eight weeks after delivery of the final X-ray dose (not shown).

To ensure that the disruption of cell proliferation was limited to the hippocampus, we performed a quantitative analysis of BrdU labeling in the SVZ immediately rostral to the hippocampus (see methods). FIG. 5D shows that the number of BrdU-positive cells in the SVZ (interaural 3.34) is unaltered by irradiation, indicating that exposure to X-rays was confined to the hippocampus and a small portion of the overlying and underlying structures. We also examined the effects of fluoxetine on cell proliferation in both the SGZ and SVZ after irradiation. As shown earlier, chronic treatment with fluoxetine increased cell proliferation in the SGZ of non-irradiated mice. In contrast, fluoxetine had no effect in the SVZ, consistent with previous reports (Malberg et al., 2000), or in irradiated animals (FIG. 5D).

Irradiation Suppresses the Response to Chronic Antidepressants In Two Behavioral Paradigms: Novelty-Suppressed Feeding and Chronic Unpredictable Stress

To test whether the radiological blockade of hippocampal neurogenesis alters the behavioral effects of ADs, we subjected adult mice to X-rays as described above, and concurrently began treatment with fluoxetine, imipramine, or vehicle before assessing their performance in the NSF test (FIGS. 5C and 6A,B). Similar to the previous observations with WT mice, treatment of sham animals with chronic fluoxetine or imipramine produced a significant reduction in the latency to feed. In contrast, these drugs had no such anxiolytic-like effect in irradiated animals. FIG. 6A also shows that the irradiation procedure did not affect the latency to feed in vehicle-treated animals. In addition, no overall change in food intake as a result of irradiation or drug treatment was detected in the food consumption test (FIG. 6B). Therefore, the lack of effect of fluoxetine and imipramine in the irradiated mice cannot be explained by an alteration in the feeding drive of these animals, or by a change in their base-line behavior in the NSF test.

To strengthen this finding, we decided to assess whether irradiation also blocks the effects of fluoxetine in a second behavioral test that detects chronic antidepressant responses, the chronic unpredictable stress paradigm (CUS). The CUS protocol consists of the sequential application of a number of stressors, presented in a varied and unpredictable manner during 5 weeks. The continued application of these stressors produces a deterioration in the state of the coat, an impaired grooming response, and weight loss (Griebel et al., 2002; Willner et al., 1992). ADs such as fluoxetine can reverse these stress-induced effects after chronic, but not acute, administration (Griebel et al., 2002; Willner et al., 1992).

Sham and irradiated mice were subjected to five weeks of CUS, beginning two weeks after the final X-ray dose, and half of the animals in each group were administered fluoxetine (10 mg/kg/day, i.p.) from weeks 3-6. The state of each animal's fur was assessed at the end of the stress period, and assigned a score based on observations from several body regions (see methods). Fluoxetine treatment was continued for one more week, after which we measured the latency to begin grooming following application of a sucrose solution to the head of each animal. As shown in FIG. 6C, fluoxetine significantly improved the state of the fur in sham animals, and this effect was absent in irradiated animals. Likewise, grooming latency was decreased by fluoxetine in sham, but not in irradiated mice (FIG. 6D). Fluoxetine-treated sham animals also displayed a trend to reverse the weight loss induced by stress, and irradiation blocked this effect (data not shown).

In conclusion, irradiation suppresses the behavioral effects of chronic fluoxetine treatment in both NSF and CUS tests, consistent with the hypothesis that hippocampal neurogenesis plays a significant role in mediating the antidepressant action of this drug.

Irradiation Does Not Alter Hippocampal Morphology, Neuroendocrine Response to Stress, Cued Fear Conditioning, or CA3/CA1 LTP

In order to evaluate the effects of irradiation on gross brain morphology and cell density within the DG, we subjected an independent group of mice to three X-ray exposures as described above. Four weeks later, no discernible changes in brain structure or integrity were found, as assessed by Nissl-staining (not shown). In addition, stereological cell counts were conducted within the superior and inferior blades of the dentate gyrus. This analysis revealed no differences between irradiated and sham animals in either the number of granule cells (291.6±20.3 and 304.6±30.7, mean±SEM in sham and irradiated, respectively; n=5) or DG volume (24.8±2.4 and 24.4±1.0×10⁷ μm³, mean±SEM in sham and irradiated, respectively; n=5).

The brain area targeted by irradiation includes not only the hippocampus, but also structures that are known to be involved in fear and anxiety responses, such as the hypothalamus and the amygdala. To rule out the possibility that the behavioral effects observed after irradiation result from damage to these structures, rather than from a blockade of neurogenesis in the hippocampus, we conducted two control experiments. First, the neuroendocrine response to stress was assessed by measuring serum corticosterone before and after open field stress. As shown in FIG. 7A, irradiated mice displayed no difference in baseline serum corticosterone, and sham and irradiated mice responded to the stressor with a comparable increase. This indicates that basal and stress-induced hypothalamic functions remain intact after irradiation.

We next examined the effects of the irradiation procedure on amygdala function using a cued fear conditioning paradigm (LeDoux et al., 1990). In this test, mice are trained to associate a conditioned stimulus (CS, tone) with an unconditioned stimulus (US, electric shock) that elicits a freezing response. 24 hours later, conditioning is measured by scoring the amount of time spent freezing during exposure to the CS alone. This association has been shown to require an intact amygdala (LeDoux et al., 1990). As shown in FIG. 7B, sham and irradiated mice showed no significant difference in the percent of time spent freezing either before or during the CS. In addition, neither group displayed any freezing to the first CS on the training day, indicating that freezing to the CS during the test period was a result of the association with the US (data not shown). These data demonstrate that irradiation does not disrupt amygdala-dependent fear conditioning.

To determine whether irradiation alters the function of mature differentiated neurons, we assessed synaptic transmission and plasticity in the CA1 region of the hippocampus. Synaptic transmission was evaluated by measuring input-output relationships between the Shaffer collateral pathway and CA1 neurons in hippocampal slices from sham and irradiated mice. We found that the slope of stimulus-response curves of field excitatory postsynaptic potentials (fEPSP) versus a wide range of stimulus intensities did not differ between sham and irradiated mice (FIG. 7C). The same slices were next tested for their ability to undergo long-term potentiation (LTP), a long-lasting enhancement of synaptic efficacy. As shown in FIG. 7D, theta-burst-induced LTP in the CA1 region was not altered in irradiated mice when compared with sham controls. These results suggest that basal synaptic transmission and plasticity in the CA1 region of the hippocampus remain normal in irradiated mice. Additionally, these physiological properties of the Schaffer collateral/CA1 connection do not appear to be impacted by a loss of neurogenesis for a period of one month.

Taken together, the results of this set of control experiments indicate that, at a time after irradiation when AD-induced behavioral responses are blocked, several structures that have been directly exposed to X-rays retain their normal functional capabilities. Therefore, the lack of AD-induced behavioral effects following irradiation does not appear to be the consequence of an impairment of hypothalamic or amygdala function, but likely results from impaired neurogenesis within the hippocampus. However, we currently cannot exclude the possibility that other, more subtle, effects of irradiation contribute to the lack of effect of antidepressants.

Discussion

Activation of 5-HT1A Receptors Contributes to the Antidepressant Effects of SSRIs

SSRIs such as fluoxetine increase extracellular levels of serotonin and activate, as a result, several of the fourteen known 5-HT receptor subtypes. While it is not known which of these receptors mediate(s) the therapeutic effects of SSRIs, several lines of evidence point to the 5-HT_{1A} receptor as a potential candidate. These include clinical trials showing that 5-HT_{1A} agonists such as buspirone are effective in the treatment of anxiety and mood disorders (Gorman, 2002), and preclinical studies, using both knockout mice and pharmacological agents, demonstrating that 5-HT_{1A} receptors modulate anxiety and depression-like behaviors (Heisler et al., 1998; Menard and Treit, 1998; Parks et al., 1998; Ramboz et al., 1998).

In order to study the contribution of this receptor to the therapeutic action of ADs, we tested mice lacking the 5-HT_{1A} receptor in the NSF paradigm. This conflict test is based on novelty-induced suppression of feeding, a phenomenon also known as “hyponeophagia” (Shephard and Broadhurst, 1982). The data indicates that, in mice, the latency to feed in a novel environment is decreased specifically by chronic, but not acute, treatment with antidepressants that act through either serotonergic (fluoxetine and imipramine) or noradrenergic (imipramine and desipramine) mechanisms (FIG. 1B). In the same paradigm, a similar decrease in the latency to feed was obtained by chronic activation of 5-HT_{1A} receptors with the direct agonist 8-OH-DPAT (FIG. 4A). Interestingly, in the NSF, 5-HT_{1A} receptor KO mice responded to the tricyclic antidepressants (TCAs) imipramine and desipramine, but not to the SSRI fluoxetine. These findings suggest that activation of 5-HT_{1A} receptors is a critical component in the mechanism of action of SSRIs. Furthermore, the finding that 5-HT_{1A} knockout mice are still responsive to TCAs that work, at least in part, through a noradrenergic mechanism indicates that SSRIs and TCAs act via independent molecular pathways. Indeed, there is both preclinical and clinical evidence supporting this interpretation (Delgado et al., 1999; Page et al., 1999).

Hippocampal Neurogenesis is Required to Mediate the Effects of Antidepressants

Since antidepressants and 5-HT_{1A} agonists display therapeutic effects only after chronic administration, these drugs likely work by eliciting gradual changes in brain circuitry, such as growth-related events. The hippocampus, one of the main sites of 5-HT_{1A} receptor expression, is capable of considerable cellular and molecular plasticity in response to various environmental and pharmacological challenges (D'Sa and Duman, 2002; McEwen, 1999). In this brain structure, new granule cells are generated during adulthood, and this process can be modulated by a number of different stimuli. Stress, aging and glucocorticoids reduce hippocampal neurogenesis while enriched environment, exercise, learning, dietary restrictions, estrogens, and various antidepressant treatments enhance it (Gould and Gross, 2002). Among the latter, SSRIs, TCAs, substance P receptor antagonists, electroconvulsive therapy, rolipram, and lithium have all been shown to stimulate neurogenesis (Chen et al., 2000; D'Sa and Duman, 2002; Malberg et al., 2000; Nakagawa et al., 2002; Rupniak, 2002). The changes in neurogenesis elicited by various classes of antidepressants require a regimen of administration that is longer than 5 days (Malberg et al., 2000 and this study), and it takes several more days for newly generated progenitors to differentiate into functional hippocampal granule cells (Hastings and Gould, 1999; van Praag et al., 2002). This lag period is reminiscent of the time required for antidepressant therapies to have appreciable clinical effects.

After confirming that a chronic treatment with two classes of ADs was able to enhance hippocampal neurogenesis in wild-type mice, we showed that mutant mice lacking the 5HT_{1A} receptor failed to respond to the neurogenic effect of SSRIs, while TCAs were still effective. Furthermore, we found that direct activation of 5-HT_{1A} receptors was sufficient to enhance hippocampal neurogenesis. These data establish a striking correlation between the ability of chronic antidepressants and 5-HT_{1A} agonists to modify behavior and their ability to stimulate neurogenesis, and suggest that the generation of new hippocampal granule cells is required for the action of antidepressant drugs.

To further test this idea, we developed an X-irradiation procedure that selectively ablates hippocampal neurogenesis, and showed that this manipulation blocks the effects of chronic fluoxetine in two behavioral paradigms. These results suggest that fluoxetine requires an increase in hippocampal neurogenesis to produce an antidepressant-like effect in the NSF and CUS tests (FIG. 6). In addition, we have shown that irradiated mice display normal fear responses in a series of behavioral tests such as the open field and the cued fear conditioning paradigms. Therefore, the lack of effect of fluoxetine and imipramine in irradiated mice is unlikely to result from a gross impairment in the circuits underlying normal emotional responses.

Although the data demonstrates a strong correspondence between behavior and neurogenesis, we found two instances of dissociation which are worthy of note: 1) 5-HT_{1A} KO mice show higher levels of anxiety-related behaviors in the NSF test, as well as in a number of other conflict tests (FIG. 3A and (Ramboz et al., 1998), but have WT levels of basal neurogenesis; 2) a 28-day ablation of neurogenesis in vehicle-treated mice does not produce any behavioral deficit in either the NSF or CUS test (FIG. 6). Concerning the first point, we have previously shown that the anxious-like phenotype of the 5-HT_{1A} KO results from the lack of expression of this receptor during the early postnatal period, (Gross et al., 2002); therefore, it is likely that the mechanisms underlying this phenotype are developmentally determined and independent of adult hippocampal neurogenesis. Regarding the second point, it is possible that a longer period of ablation is necessary to reveal behavioral deficits in the NSF and CUS paradigms, and thereby uncover a potential role of basal hippocampal neurogenesis. Alternatively, neurons that are generated in response to antidepressants may have different functional properties than cells generated in base-line conditions. In either case, the lack of effect of irradiation on basal behavioral responses in the NSF and CUS, as well as in the Open Field test (not shown), suggests that the focal X-ray procedure does not elicit a non-specific behavioral impairment and strengthens the hypothesis that neurogenesis contributes to the effects of antidepressants.

A Potential Role for Hippocainpal Neurogenesis in Mood and Anxiety Regulation

The results of these studies are surprising for two reasons: first, the number of cells generated in the adult hippocampus each month, both in normal conditions and after treatment with ADs, is relatively low when compared to the total number of neurons in this structure (Cameron and McKay, 2001) and, second, these results suggest a novel role for the hippocampus in modulating anxiety-like behaviors.

Newly generated neurons have been suggested to exhibit electrophysiological properties and patterns of connectivity similar to those of immature hippocampal granular neurons. Specifically, young neurons born in the adult hippocampus may transiently form a greater number of connections than older granule cells, recapitulating normal developmental processes (Gould and Gross, 2002). Also, due to a reduced GABA inhibition, plastic changes such as long-term potentiation (LTP) may be more readily evoked in new neurons than in mature granule cells (Snyder et al., 2001; Wang et al., 2000). It is therefore possible that a small number of new cells could significantly influence signaling within the hippocampus (Henze et al., 2002).

While the hippocampus has long been associated with learning and memory processes, there is increasing evidence that this structure is also involved in the modulation of emotional responses (Eichenbaum, 1999; Gray, 2000; Kempermann, 2002; McNaughton, 1997). Lesions of the ventral hippocampus or local administration of pharmacological agents into the dorsal and ventral hippocampus result in altered behavior in a number of rodent models of anxiety (Deacon et al., 2002; Degroot and Treit, 2002; File et al., 2000; Gray, 2000; Menard and Treit, 2001; Menard and Treit, 1998). Recently, a double dissociation was found regarding the roles of the dorsal and ventral hippocampus in spatial learning versus hyponeophagia, an anxiety test that is similar to the Novelty-suppressed feeding test used in the present study. Specifically, Bannerman and coworkers demonstrated that while dorsal hippocampal lesions had an effect on spatial learning, but not on hyponeophagia, ventral lesions decreased hyponeophagia but had no effect on learning (Bannerman et al., 2002). Thus, a functional differentiation of the hippocampus may exist along its dorso-ventral axis.

In further support of the hippocampus's involvement in mood regulation are recent reports that manipulations of transcription or neurotrophic factors in this structure can produce an antidepressant-like effect (Nakagawa et al., 2002; Shirayama et al., 2002). In addition, hippocampal glucocorticoid receptors have been shown to be involved in the negative feedback loop of stress hormones on the hypothalamus-pituitary-adrenal axis (Jacobson and Sapolsky, 1991). Finally, there is evidence in both the rodent and the human literature that chronic stress and depression result in hippocampal atrophy, and that these effects can be reversed by certain antidepressants (Czeh et al., 2001; McEwen, 1999; Moore et al., 2000).

Molecular Mechanisms of Antidepressant Action

We are currently attempting to uncover the molecular mechanisms governing the increases in hippocampal neurogenesis elicited by both direct and indirect 5-HT_{1A} agonists. 5-HT_{1A} receptors are expressed on serotonergic raphe neurons, where they act as inhibitory autoreceptors, and in postsynaptic structures, including the hippocampus. Unlike hippocampal 5-HT_{1A} receptors, raphe autoreceptors have been shown to desensitize after chronic antidepressant treatment (Blier and de Montigny, 1994). Therefore, the effects of chronic ADs are more likely to be mediated by postsynaptic 5-HT_{1A} receptors. Among the postsynaptic sites, 5-HT_{1A} receptors are most abundant on CA1 pyramidal neurons, but are virtually absent from the dentate gyrus in mice (Gross et al., 2002). Based on this pattern of expression, it is unlikely that the neurogenic effect of 5-HT_{1A} agonists results from a direct activation of these receptors on hippocampal progenitor cells located in the SGZ. It is more plausible that activation of 5-HT_{1A} receptors triggers the release of growth factors that will then stimulate the division and/or differentiation of hippocampal progenitor cells. In support of this argument are a number of studies showing that growth factors, including BDNF, NT3, IGF1 and FGF2, stimulate neurogenesis both in vitro and in vivo (Aberg et al., 2000; Benraiss et al., 2001; Kirschenbaum and Goldman, 1995; Pencea et al., 2001; Yoshimura et al., 2001). In addition, BDNF mRNA levels increase in the hippocampus in response to antidepressants (Nibuya et al., 1995), and both BDNF and NT-3 can have antidepressant-like effects when injected into the hippocampus (Shirayama et al., 2002). In keeping with this hypothesis, we have recently shown that chronic treatment with a 5-HT_{1A} agonist increases BDNF mRNA levels in the hippocampus (data not shown).

References

Aberg, M. A., Aberg, N. D., Hedbacker, H., Oscarsson, J., and Eriksson, P. S. (2000). Peripheral infusion of IGF-I selectively induces neurogenesis in the adult rat hippocampus. J Neurosci 20, 2896-903.

Altman, J. (1962). Are new neurons formed in the brains of adult mammals? Science 135, 1127-1128.

Altman, J., and Das, G. D. (1965). Autoradiographic and histological evidence of postnatal hippocampal neurogenesis in rats. J Comp Neurol 124, 319-335.

Altman, J., and Das, G. D. (1966). Autoradiographic and histological studies of postnatal neurogenesis. I. A longitudinal investigation of the kinetics, migration and transformation of cells incorporating tritiated thymidine in neonate rats, with special reference to postnatal neurogenesis in some brain regions. J Comp Neurol 126.

Bannerman, D. M., Deacon, R. M., Offen, S., Friswell, J., Grubb, M., and Rawlins, J. N. (2002). Double dissociation of function within the hippocampus: spatial memory and hyponeophagia. Behav Neurosci 116, 884-901.

Benraiss, A., Chmielnicki, E., Lerner, K., Roh, D., and Goldman, S. A. (2001). Adenoviral brain-derived neurotrophic factor induces both neostriatal and olfactory neuronal recruitment from endogenous progenitor cells in the adult forebrain. J Neurosci 21, 6718-31.

Blanchard, R. J., and Blanchard, D. C. (1969). Crouching as an index of fear. J Comp Physiol Psychol 67, 370-5.

Blanchard, R. J., and Blanchard, D. C. (1969). Passive and active reactions to fear-eliciting stimuli. J Comp Physiol Psychol 68, 129-35.

Blier, P., and de Montigny, C. (1994). Current advances and trends in the treatment of depression. Trends Pharmacol Sci 15, 220-6.

Bodnoff, S. R., Suranyi-Cadotte, B., Aitken, D. H., Quirion, R., and Meaney, M. J. (1988). The effects of chronic antidepressant treatment in an animal model of anxiety. Psychopharmacology 95, 298-302.

Bodnoff, S. R., Suranyi-Cadotte, B., Quirion, R., and Meaney, M. J. (1989). A comparison of the effects of diazepam versus several typical and atypical anti-depressant drugs in an animal model of anxiety. Psychopharmacology 97, 277-9.

Bremner, J. D., Randall, P., Vermetten, E., Staib, L., Bronen, R. A., Mazure, C., Capelli, S., McCarthy, G., Innis, R. B., and Charney, D. S. (1997). Magnetic resonance imaging-based measurement of hippocampal volume in posttraumatic stress disorder related to childhood physical and sexual abuse—a preliminary report. Biol Psychiatry 41, 23-32.

Cameron, H. A., and McKay, R. D. (2001). Adult neurogenesis produces a large pool of new granule cells in the dentate gyrus. J Comp Neurol 435, 406-17.

Chen, G., Rajkowska, G., Du, F., Seraji-Bozorgzad, N., and Manji, H. K. (2000). Enhancement of hippocampal neurogenesis by lithium. J Neurochem 75, 1729-34.

Cryan, J. F., Markou, A., and Lucki, I. (2002). Assessing antidepressant activity in rodents: recent developments and future needs. Trends Pharmacol Sci 23, 238-45.

Czeh, B., Michaelis, T., Watanabe, T., Frahm, J., de Biurrun, G., van Kampen, M., Bartolomucci, A., and Fuchs, E. (2001). Stress-induced changes in cerebral metabolites, hippocampal volume, and cell proliferation are prevented by antidepressant treatment with tianeptine. Proc Natl Acad Sci U S A 98, 12796-801.

D'Sa, C., and Duman, R. S. (2002). Antidepressants and neuroplasticity. Bipolar Disord 4, 183-94.

Deacon, R. M., Bannerman, D. M., and Rawlins, J. N. (2002). Anxiolytic effects of cytotoxic hippocampal lesions in rats. Behav Neurosci 116, 494-7.

Degroot, A., and Treit, D. (2002). Dorsal and ventral hippocampal cholinergic systems modulate anxiety in the plus-maze and shock-probe tests. Brain Res 949, 60.

Delgado, P. L., Miller, H. L., Salomon, R. M., Licinio, J., Krystal, J. H., Moreno, F. A., Heninger, G. R., and Charney, D. S. (1999). Tryptophan-depletion challenge in depressed patients treated with desipramine or fluoxetine: implications for the role of serotonin in the mechanism of antidepressant action. Biol Psychiatry 46, 212-20.

Doetsch, F., Caille, I., Lim, D. A., Garcia-Verdugo, J. M., and Alvarez-Buylla, A. (1999). Subventricular zone astrocytes are neural stem cells in the adult mammalian brain. Cell 97, 703-16.

Duffy, S. N., Craddock, K. J., Abel, T., and Nguyen, P. V. (2001). Environmental enrichment modifies the PKA-dependence of hippocampal LTP and improves hippocampus-dependent memory. Learn Mem 8, 26-34.

Duman, R. S., and Charney, D. S. (1999). Cell atrophy and loss in major depression. Bill Psychiatry 45, 1083-4.

Duman, R. S., Heninger, G. R., and Nestler, E. J. (1997). A molecular and cellular theory of depression. Arch Gen Psychiatry 54, 597-606.

Eichenbaum, H. (1999). The hippocampus: The shock of the new. Curr Biol 9, R482-4.

Feng, R., Rampon, C., Tang, Y. P., Shrom, D., Jin, J., Kyin, M., Sopher, B., Miller, M. W., Ware, C. B., Martin, G. M., Kim, S. H., Langdon, R. B., Sisodia, S. S., and Tsien, J. Z. (2001). Deficient neurogenesis in forebrain-specific presenilin-1 knockout mice is associated with reduced clearance of hippocampal memory traces. Neuron 32, 911-26.

File, S. E., Kenny, P. J., and Cheeta, S. (2000). The role of the dorsal hippocampal serotonergic and cholinergic systems in the modulation of anxiety. Pharmacol Biochem Behav 66, 65-72.

Frank-Kamenetsky, M., Zhang, X., Bottega, S., Guicherit, O., Wichterle, H., Dudek, H., Bumcort, D., Wang, F., Jones, S., Shulok, J., Rubin, L., and Porter, J. (2002). Small-molecule modulators of Hedgehog signaling: identification and characterization of Smoothened agonists and antagonists. J. Biol. 1:10, 10.1-9.

Garcia-Verdugo, J. M., Doetsch, F., Wichterle, H., Lim, D. A., and Alvarez-Buylla, A. (1998). Architecture and cell types of the adult subventricular zone: in search of the stem cells. J Neurobiol 36, 234-48.

Gorman, J. M. (2002). Treatment of generalized anxiety disorder. J Clin Psychiatry 63, 17-23.

Gould, E., and Gross, C. G. (2002). Neurogenesis in adult mammals: some progress and problems. 3 Neurosci 22, 619-23.

Gould, E., and Tanapat, P. (1999). Stress and hippocampal neurogenesis. Biol Psychiatry 46, 1472-9.

Gould, E., Tanapat, P., McEwen, B. S., Flugge, G., and Fuchs, E. (1998). Proliferation of granule cell precursors in the dentate gyrus of adult monkeys is diminished by stress. Proc Natl Acad Sci U S A 95, 3168-71.

Gray, J. A., & MaNaughton, N. (2000). The Neuropsychology of Anxiety (2nd ed.) (Oxford, England: Oxford University Press).

Griebel, G., Simiand, J., Serradeil-Le Gal, C., Wagnon, J., Pascal, M., Scatton, B., Maffrand, J. P., and Soubrie, P. (2002). Anxiolytic- and antidepressant-like effects of the non-peptide vasopressin V1b receptor antagonist, SSR149415, suggest an innovative approach for the treatment of stress-related disorders. Proc Natl Acad Sci U S A 99, 6370-5.

Gross, C., Santarelli, L., Brunner, D., Zhuang, X., and Hen, R. (2000). Altered fear circuits in 5-HT(1A) receptor KO milce. Biol Psychiatry 48, 1157-63.

Gross, C., Zhuang, X., Stark, K., Ramboz, S., Oosting, R., Kirby, L., Santarelli, L., Beck, S., and Hen, R. (2002). SerotoninlA receptor acts during development to establish normal anxiety-like behaviour in the adult. Nature 416, 396-400.

Gurvits, T. V., Shenton, M. E., Hokama, H., Ohta, H., Lasko, N. B., Gilbertson, M. W., Orr, S. P., Kikinis, R., Jolesz, F. A., McCarley, R. W., and Pitman, R. K. (1996). Magnetic resonance imaging study of hippocampal volume in chronic, combat-related posttraumatic stress disorder. Biol Psychiatry 40, 1091-9.

Hastings, N. B., and Gould, E. (1999). Rapid extension of axons into the CA3 region by adult-generated granule cells. J Comp Neurol 413, 146-54.

Heisler, L. K., Chu, H. M., Brennan, T. J., Danao, J. A., Bajwa, P., Parsons, L. H., and Tecott, L. H. (1998). Elevated anxiety and antidepressant-like responses in serotonin 5-HT1A receptor mutant mice. Proc Natl Acad Sci U S A 95, 15049-54.

Henze, D. A., Wittner, L., and Buzsaki, G. (2002). Single granule cells reliably discharge targets in the hippocampal CA3 network in vivo. Nat Neurosci 5, 790-5.

Jacobson, L., and SapQlsky, R. (1991). The role of the hippocampus in feedback regulation of the hypothalamic-pituitary-adrenocortical axis. Endocr Rev 12, 118-34.

Kaplan, M. S., and Hinds, J. W. (1977). Neurogenesis in the adult rat: electron microscopic analysis of light radioautographs. Science 197, 1092-4.

Kempermann, G. (2002). Regulation of adult hippocampal neurogenesis—implications for novel theories of major depression. Bipolar Disord 4, 17-33.

Kempermann, G., Kuhn, H. G., and Gage, F. H. (1997). Genetic influence on neurogenesis in the dentate gyrus of adult mice. Proc Natl Acad Sci U S A 94, 10409-14.

Kempermann, G., Kuhn, H. G., and Gage, F. H. (1997). More hippocampal neurons in adult mice living in an enriched environment. Nature 386, 493-5.

Kirschenbaum, B., and Goldman, S. A. (1995). Brain-derived neurotrophic factor promotes the survival of neurons arising from the adult rat forebrain subependymal zone. Proc Natl Acad Sci U S A 92, 210-4.

Kopp, C., Vogel, E., and Misslin, R. (1999). Comparative study of emotional behaviour in three inbred strains of mice. Behavioural Processes 47, 161-74.

Kuhn, H. G., Dickinson-Anson, H., and Gage, F. H. (1996). Neurogenesis in the dentate gyrus of the adult rat: age-related decrease of neuronal progenitor proliferation. J Neurosci 16, 2027-33.

Lai, K., Kaspar, B., Gage, F., and Schaffer, D. (2003). Sonic hedgehog upregulates adult neural progenitor proliferation in vitro and in vivo. Nature Neuroscience Vol.6 No.1, 21-27.

LeDoux, J. E., Cicchetti, P., Xagoraris, A., and Romanski, L. M. (1990). The lateral amygdaloid nucleus: sensory interface of the amygdala in fear conditioning. J Neurosci 10, 1062-9.

LeDoux, J. E., Sakaguchi, A., and Reis, D. J. (1984). Subcortical efferent projections of the medial geniculate nucleus mediate emotional responses conditioned to acoustic stimuli. J Neurosci 4, 683-98.

Liberatore, G. T., Jackson-Lewis, V., Vukosavic, S., Mandir, A. S., Vila, M., McAuliffe, W. G., Dawson, V. L., Dawson, T. M., and Przedborski, S. (1999). Inducible nitric oxide synthase stimulates dopaminergic neurodegeneration in the MPTP model of Parkinson disease. Nat Med 5, 1403-9.

Malberg, J. E., Eisch, A. J., Nestler, E. J., and Duman, R. S. (2000). Chronic antidepressant treatment increases neurogenesis in adult rat hippocampus. J Neurosci 20, 9104-10.

Manev, H., Uz, T., Smalheiser, N. R., and Manev, R. (2001). Antidepressants alter cell proliferation in the adult brain in vivo and in neural cultures in vitro. Eur J Pharmacol 411, 67-70.

Masand, P. S., and Gupta, S. (1999). Selective serotonin-reuptake inhibitors: an update. Harv Rev Psychiatry 7, 69-84.

McEwen, B. S. (1999). Stress and hippocampal plasticity. Annu Rev Neurosci 22, 105-22.

McNaughton, N. (1997). Cognitive dysfunction resulting from hippocampal hyperactivity—a possible cause of anxiety disorder? Pharmacol Biochem Behav 56, 603-11.

Menard, J., and Treit, D. (2001). The anxiolytic effects of intra-hippocampal midazolam are antagonized by intra-septal L-glutamate. Brain Res 888, 163-166.

Menard, J., and Treit, D. (1998). The septum and the hippocampus differentially mediate anxiolytic effects of R(+)-8-OH-DPAT. Behav Pharmacol 9, 93-101.

Moore, G. J., Bebchuk, J. M., Wilds, I. B., Chen, G., Manji, H. K., and Menji, H. K. (2000). Lithium-induced increase in human brain grey matter. Lancet 356, 1241-2.

Nakagawa, S., Kim, J. E., Lee, R., Malberg, J. E., Chen, J., Steffen, C., Zhang, Y. J., Nestler, E. J., and Duman, R. S. (2002). Regulation of neurogenesis in adult mouse hippocampus by cAMP and the cAMP response element-binding protein. J Neurosci 22, 3673-82.

Nibuya, M., Morinobu, S., and Duman, R. S. (1995). Regulation of BDNF and trkB mRNA in rat brain by chronic electroconvulsive seizure and antidepressant drug treatments. J Neurosci 15, 7539-47.

Nilsson, M., Perfilieva, E., Johansson, U., Orwar, O., and Eriksson, P. S. (1999). Enriched environment increases neurogeniesis in the adult rat dentate gyrus and improves spatial memory. J Neurobiol 39, 569-78.

Page, M. E., Detke, M. J., Dalvi, A., Kirby, L. G., and Lucki, I. (1999). Serotonergic mediation of the effects of fluoxetine, but not desipramine, in the rat forced swimming test. Psychopharmacology (Berl) 147, 162-7.

Parks, C. L., Robinson, P. S., Sibille, E., Shenk, T., and Toth, M. (1998). Increased anxiety of mice lacking the serotoninlA receptor. Proc Natl Acad Sci U S A 95, 10734-9.

Pencea, V., Bingaman, K. D., Wiegand, S. J., and Luskin, M. B. (2001). Infusion of brain-derived neurotrophic factor into the lateral ventricle of the adult rat leads to new neurons in the parenchyma of the striatum, septum, thalamus, and hypothalamus. J Neurosci 21, 6706-17.

Pham, T. M., Soderstrom, S., Winblad, B., and Mohammed, A. H. (1999). Effects of environmental enrichment on cognitive function and hippocampal NGF in the non-handled rats. Behav Brain Res 103, 63-70.

Phillips, T. J., Hen, R., and Crabbe, J. C. (1999). Complications associated with genetic background effects in research using knockout mice. Psychopharmacology (Bern) 147, 5-7.

Ramboz, S., Oosting, R., Amara, D. A., Kung, H. F., Blier, P., Mendelsohn, M., Mann, J. J., Brunner, D., and Hen, R. (1998). Serotonin receptor 1A knockout: an animal model of anxiety-related disorder. Proc Natl Acad Sci U S A 95, 14476-81.

Rupniak, N. M. (2002). Elucidating the antidepressant actions of substance P .(NK1 receptor) antagonists. Curr Opin Investig Drugs 3, 257-61.

Santarelli, L., Gobbi, G., Debs, P. C., Sibille, E. T., Blier, P., Hen, R., and Heath, M. J. (2001). Genetic and pharmacological disruption of neurokinin 1 receptor function decreases anxiety-related behaviors and increases serotonergic function. Proc Natl Acad Sci U S A 98, 1912-7.

Sapolsky, R. (1992). Stress, the Aging Brain and the Mechanism of Neuron Death. (Cambridge, MA: Mass. Inst. Technol).

Sargent, P. A., Kjaer, K. H., Bench, C. J., Rabiner, E. A., Messa, C., Meyer, J., Gunn, R. N., Grasby, P. M., and Cowen, P. J. (2000). Brain serotonin1A receptor binding measured by positron emission tomography with [11C]WAY-100635: effects of depression and antidepressant treatment. Arch Gen Psychiatry 57, 174-80.

Seki, T., and Arai, Y. (1995). Age-related production of new granule cells in the adult dentate gyrus. Neuroreport 6, 2479-82.

Seri, B., Garcia-Verdugo, J. M., McEwen, B. S., and Alvarez-Buylla, A. (2001). Astrocytes give rise to new neurons in the adult mammalian hippocampus. J Neurosci 21, 7153-60.

Shah, P. J., Ebmeier, K. P., Glabus, M. F., and Goodwin, G. M. (1998). Cortical grey matter reductions associated with treatment-resistant chronic unipolar depression. Controlled magnetic resonance imaging study. Br J Psychiatry 172, 527-32.

Sheline, Y. I., Wang, P. W., Gado, M. H., Csernansky, J. G., and Vannier, M. W. (1996). Hippocampal atrophy in recurrent major depression. Proc Natl Acad Sci U S A 93, 3908-13.

Shephard, R. A., and Broadhurst, P. L. (1982). Hyponeophagia and arousal in rats: effects of diazepam, 5-methoxy-N,N-dimethyltryptamine, d-amphetamine and food deprivation. Psychopharmacology 78, 368-72.

Shirayama, Y., Chen, A. C., Nakagawa, S., Russell, D. S., and Duman, R. S. (2002). Brain-derived neurotrophic factor produces antidepressant effects in behavioral models of depression. J Neurosci 22, 3251-61.

Shors, T. J., Miesegaes, G., Beylin, A., Zhao, M., Rydel, T., and Gould, E. (2001). Neurogenesis in the adult is involved in the formation of trace memories. Nature 410, 372-6.

Snyder, J. S., Kee, N., and Wojtowicz, J. M. (2001). Effects of adult neurogenesis on synaptic plasticity in the rat dentate gyrus. J Neurophysiol 85, 2423-31.

Stein, M. B., Koverola, C., Hanna, C., Torchia, M. G., and McClarty, B. (1997). Hippocampal volume in women victimized by childhood sexual abuse. Psychol Med 27, 951-9.

Tada, E., Parent, J. M., Lowenstein, D. H., and Fike, J. R. (2000). X-irradiation causes a prolonged reduction in cell proliferation in the dentate gyrus of adult rats. Neuroscience 99, 33-41.

van Praag, H., Schinder,. A. F., Christie, B. R., Toni, N., Palmer, T. D., and Gage, F. H. (2002). Functional neurogenesis in the adult hippocampus. Nature 415, 1030-4.

Vitolo, 0. V., Sant'Angelo, A., Costanzo, V., Battaglia, F., Arancio, O., and Shelanski, M. (2002). Amylbid beta—peptide inhibition of the PKA/CREB pathway and long-term potentiation: Reversibility by drugs that enhance cAMP signaling. Proc Natl Acad Sci U S A 99, 13217-21.

Wang, S., Scott, B. W., and Wojtowicz, J. M. (2000). Heterogenous properties of dentate granule neurons in the adult rat. J Neurobiol 42, 248-57.

Williams, B. M., Luo, Y., Ward, C., Redd, K., Gibson, R., Kuczaj, S. A., and McCoy, J. G. (2001). Environmental enrichment: effects on spatial memory and hippocampal CREB immunoreactivity. Physiol Behav 73, 649-58.

Willner, P., Muscat, R., and Papp, M. (1992). Chronic mild stress-induced anhedonia: a realistic animal model of depression. Neurosci Biobehav Rev 16, 525-34.

Wong, M. L., and Licinio, J. (2001). Research and treatment approaches to depression. Nat Rev Neurosci 2, 343-51.

Yoshimura, S., Takagi, Y., Harada, J., Teramoto, T., Thomas, S. S., Waeber, C., Bakowska, J. C., Breakefield, X. O., and Moskowitz, M. A. (2001). FGF-2 regulation of neurogenesis in adult hippocampus after brain injury. Proc Natl Acad Sci U S A 98, 5874-9.

1. A method for determining whether an agent increases brain progenitor cell division comprising: (i) administering,the agent to a non-human subject; and (ii) determining whether the resulting brain progenitor cell division in the subject is greater than that in a subject to which the agent was not administered, thereby determining whether the agent increases brain progenitor cell division.

2-27. (canceled)

28. A method for treating anxiety, depression, a cognitive disorder or a neuro-degenerative disorder by administering to an afflicted subject a therapeutically effective amount of an agent determined to have the ability to increase brain progenitor cell division, wherein such ability is determined by a method comprising (i) administering the agent to a non-human subject, and (ii) determining whether the resulting brain progenitor cell division in the subject is greater than that in a subject to which the agent was not administered.

29-34. (canceled)

35. A method for inhibiting the onset of anxiety, depression or a cognitive disorder by administering to a subject in need thereof a prophylactically effective amount of an agent determined as having the ability to increase brain progenitor cell division, wherein such ability is determined by a method comprising (i) administering the agent to a non-human subject, and (ii) determining whether the resulting brain progenitor cell division in the subject is greater than that in a subject to which the agent was not administered.

36-41. (canceled)

42. A composition comprising (a) a pharmaceutically acceptable carrier, and (b) an agent determined as having the ability to increase brain progenitor cell division, wherein such ability is determined by a method comprising (i) administering the agent to a non-human subject, and (ii) determining whether the resulting brain progenitor cell division in the subject is greater than that in a subject to which the agent was not administered.

43-47. (canceled)

48. An article of manufacture comprising a packaging material having therein an agent determined as having the ability to increase brain progenitor cell division, and a label indicating a use of the agent for inhibiting the onset of anxiety, depression, a cognitive disorder or a neurodegenerative disorder in a subject, wherein such ability is determined by a method comprising (i) administering the agent to a non-human subject, and (ii) determining whether the resulting brain progenitor cell division in the subject is greater than that in a subject to which the agent was not administered.

49-54. (canceled)

55. An article of manufacture comprising a packaging material having therein an agent determined as having the ability to increase brain progenitor cell division, and a label indicating a use of the agent for treating anxiety, depression, a cognitive disorder or a neurodegenerative disorder in a subject, wherein such ability is determined by a method comprising (i) administering the agent to a non-human subject, and (ii) determining whether the resulting brain progenitor cell division in the subject is greater than that in a subject to which the agent was not administered.

56-61. (canceled)

62. A method for treating anxiety, depression, a cognitive disorder or a neuro-degenerative disorder by administering to an afflicted subject a therapeutically effective amount of Hh-Ag 1.1, Hh-Ag 1.2, Hh-Ag 1.3, or a derivative of Hh-Ag 1.1, Hh-Ag 1.2 or Hh-Ag 1.3.

63. A method for inhibiting the onset of anxiety, depression or a cognitive disorder by administering to a subject in need thereof a prophylactically effective amount of Hh-Ag 1.1, Hh-Ag 1.2, Hh-Ag 1.3, or a derivative of Hh-Ag 1.1, Hh-Ag 1.2 or Hh-Ag 1.3.

64. A composition comprising (a) a pharmaceutically acceptable carrier, and (b) Hh-Ag 1.1, Hh-Ag 1.2, Hh-Ag 1.3, or a derivative of Hh-Ag 1.1, Hh-Ag 1.2 or Hh-Ag 1.3.

65. An article of manufacture comprising a packaging material having therein Hh-Ag 1.1, Hh-Ag 1.2, Hh-Ag 1.3, or a derivative of Hh-Ag 1.1, Hh-Ag 1.2 or Hh-Ag 1.3 and a label indicating a use of Hh-Ag 1.1, Hh-Ag 1.2, Hh-Ag 1.3, or a derivative of Hh-Ag 1.1, Hh-Ag 1.2 or Hh-Ag 1.3 for inhibiting the onset of anxiety, depression or a cognitive disorder in a subject.

66. An article of manufacture comprising a packaging material having therein Hh-Ag 1.1, Hh-Ag 1.2, Hh-Ag 1.3, or a derivative of Hh-Ag 1.1, Hh-Ag 1.2 or Hh-Ag 1.3 and a label indicating a use of Hh-Ag 1.1, Hh-Ag 1.2, Hh-Ag 1.3, or a derivative of Hh-Ag 1.1, Hh-Ag 1.2 or Hh-Ag 1.3 for treating anxiety, depression, a cognitive disorder or a neurodegenerative disorder in a subject.
